(19) United States
(12) Patent Application Publication
(10) Pub. No.: US 2026/0000398 A1
(43) Pub. Date: Jan. 1, 2026

(54) SURGICAL INSTRUMENT HAVING LINK-DRIVEN ARTICULATION JOINT

(71) Applicant: Cilag GmbH International, Zug (CH)

(72) Inventor: Paul Moubarak, West Chester, OH (US)

(21) Appl. No.: 18/756,023

(22) Filed: Jun. 27, 2024

(51) Int. Cl. A61B 17/072; A61B 17/00

(52) U.S. Cl. CPC A61B 17/07207; A61B 2017/00314; A61B 2017/00327; A61B 2017/07257; A61B 2017/07271; A61B 2017/07285

(57) ABSTRACT

A surgical instrument (1100), including: a shaft (1600) defining a longitudinal axis; an end effector (1200); and an articulation assembly (1300). The articulation assembly being positioned between the shaft and the end effector and configured to articulate the end effector relative to the shaft about each of a pitch axis and a yaw axis. The articulation assembly including: a pitch articulation beam (1354) and a yaw articulation beam (1344). Wherein the pitch articulation beam is configured to drive a pitch of the end effector relative to the shaft about the pitch axis by pushing a first linked portion of the end effector distally and pulling the first linked portion of the end effector proximally. Wherein the yaw articulation beam is configured to drive a yaw of the end effector relative to the shaft about the yaw axis by pushing a second linked portion of the end effector distally.

### BACKGROUND

In some settings, endoscopic surgical instruments may be preferred over traditional open surgical devices to minimize the size of the surgical incision as well as post-operative recovery time and complications. Consequently, some endoscopic surgical instruments may be suitable for placement of a distal end effector at a desired surgical site through the cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft that extends proximally from the end effector to a handle portion that is manipulated by the clinician, or alternatively to a robot. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Such endoscopic surgical staplers may also be used in open procedures and/or other non-endoscopic procedures. By way of example only, a surgical stapler may be inserted through a thoracotomy and thereby between a patient's ribs to reach one or more organs in a thoracic surgical procedure that does not use a trocar as a conduit for the stapler. Such procedures may include the use of the stapler to sever and close a vessel leading to an organ, such as a lung. For instance, the vessels leading to an organ may be severed and closed by a stapler before removal of the organ from the thoracic cavity. Of course, surgical staplers may be used in various other settings and procedures.

The surgical stapling features of the present disclosure seek to articulate the end effector using only two inputs related to pitch and yaw articulation. Using rigid links allows the end effector to articulate in any combination of pitch and yaw while avoiding a dearticulation response that may otherwise result from external loads applied to the end effector or lengthening of articulation cables. While various kinds of surgical staplers and associated components have been made and used, it is believed that no one prior to the inventor(s) has made or used the invention described in the appended claim

### BRIEF DESCRIPTION OF THE DRAWINGS

The accompanying drawings, which are incorporated in and constitute a part of this specification, illustrate examples of the invention, and, together with the general description of the invention given above, and the detailed description of the examples given below, serve to explain the principles of the present invention.

FIG. 1 is a perspective view of an illustrative surgical instrument having a housing, a shaft assembly, an articulation joint, and an end effector;

FIG. 2 is a partial perspective view of the surgical instrument of FIG. 1, with select components omitted from view to reveal portions of a cable articulation subsystem, a knife firing subsystem, and a roll subsystem of the surgical instrument;

FIG. 3 is an enlarged perspective view of the end effector and the articulation joint of the surgical instrument of FIG. 1;

FIG. 4 is an exploded view of a distal end portion of the surgical instrument of FIG. 1;

FIG. 5 is an enlarged perspective view of a knife of the end effector of the surgical instrument of FIG. 1;

FIG. 6 is an end view of the end effector of FIG. 3;

FIG. 7 is an enlarged perspective view of the end effector and the articulation joint of FIG. 3, with an anvil of the end effector omitted;

FIG. 8A is a side cross-sectional view of a distal end portion of the surgical instrument of FIG. 1, depicting the anvil in an open position;

FIG. 8B is a side cross-sectional view of the distal end portion of the surgical instrument of FIG. 1, depicting the anvil in a grasping position with the knife partially advanced;

FIG. 8C is a side cross-sectional view of the distal end portion of the surgical instrument of FIG. 1, depicting the anvil in a clamping position with the knife partially advanced;

FIG. 8D is a side cross-sectional view of the distal end portion of the surgical instrument of FIG. 1, depicting the anvil in the clamping position with the knife fully advanced;

FIG. 9A is an enlarged side cross-sectional view of a proximal end portion of the end effector of the surgical instrument of FIG. 1, depicting the anvil in the open position;

FIG. 9B is an enlarged side cross-sectional view of the proximal end portion of the end effector of the surgical instrument of FIG. 1, depicting the anvil in a grasping position with the knife partially advanced;

FIG. 9C is an enlarged side cross-sectional view of the proximal end portion of the end effector of the surgical instrument of FIG. 1, depicting the anvil in a clamping position with the knife partially advanced;

FIG. 9D is an enlarged side cross-sectional view of the proximal end portion of the end effector of the surgical instrument of FIG. 1, depicting the anvil in the clamping position with the knife fully advanced;

FIG. 10 is an exploded perspective view of the articulation joint of the surgical instrument of FIG. 1;

FIG. 11 is an end view of the articulation joint of FIG. 10;

FIG. 12 is a cross-sectional view of a portion of the articulation joint of FIG. 10, taken along line 12-12 in FIG. 11;

FIG. 13 is a cross-sectional view of a portion of the articulation joint of FIG. 10, taken along line 13-13 in FIG. 11;

FIG. 14 is a perspective view of the distal end of the surgical instrument of FIG. 1, depicting the end effector articulated vertically and laterally with the anvil open;

FIG. 15 is a side view of the distal end of the surgical instrument of FIG. 1, depicting the end effector articulated vertically with the anvil closed;

FIG. 16 is a top view of the distal end of the surgical instrument of FIG. 1, depicting the end effector articulated laterally with the anvil closed;

FIG. 17 is an exploded perspective view of a portion of the surgical instrument of FIG. 1, depicting portions of the cable articulation subsystem, the knife firing subsystem, and the roll subsystem;

FIG. 18 is a top view of a proximal end of the surgical instrument of FIG. 1, depicting portions of the cable articulation subsystem, the knife firing subsystem, and the roll subsystem;

FIG. 19 is a perspective view of a shaft assembly, a differential, and a firing rod of the surgical instrument of FIG. 1;

FIG. 20 is perspective view of an alternative surgical instrument having an end effector, an articulation joint, and a shaft;

FIG. 21 is a front perspective view of the surgical instrument of FIG. 20, with the outer shaft omitted from view;

FIG. 22 is a rear perspective view of the surgical instrument of FIG. 20, with being the outer shaft omitted from view;

FIG. 23 is an enlarged top perspective view of the articulation joint of FIG. 20, with the outer shaft omitted form view, showing a pair of yaw articulation beams and a pair of pitch articulation beams;

FIG. 24 is an exploded perspective view of a portion of the articulation joint of FIG. 20, with the outer shaft omitted form view, showing details of a link assembly and a constant velocity joint of the articulation joint;

FIG. 25A is a top plan view of the surgical instrument of FIG. 20, with the outer shaft omitted from view, showing the end effector in a straight, non-articulated orientation in which both yaw rack gears are longitudinally aligned with each other;

FIG. 25B1 is a plan view of a length-conservative configuration of the surgical instrument of FIG. 20 in which a yaw carrier is longitudinally fixed relative to the shaft, with the outer shaft omitted from view and showing the end effector in a yawed, articulated orientation;

FIG. 25B2 is a plan view of a non-length-conservative configuration of the surgical instrument of FIG. 20 in which the yaw carrier is longitudinally translatable relative to the shaft, with the outer shaft omitted from view and showing the end effector in a yawed, articulated orientation;

FIG. 26A is a top plan view of the surgical instrument of FIG. 20, with the outer shaft omitted from view, showing the end effector in a straight, non-articulated orientation in which the yaw rack gears are longitudinally aligned and the pitch rack gears are longitudinally aligned; and

FIG. 26B is a top plan view of the surgical instrument of FIG. 20, with the outer shaft omitted from view, showing the end effector in an articulated orientation in which the yaw rack gears are longitudinally offset and the pitch rack gears are longitudinally offset.

### DETAILED DESCRIPTION

The following detailed description should be read with reference to the drawings, in which like elements in different drawings are identically numbered. The drawings, which are not necessarily to scale, depict selected versions and are not intended to limit the scope of the invention. The detailed description illustrates by way of example, not by way of limitation, the principles of the invention. This description will clearly enable one skilled in the art to make and use the invention, and describes several versions, adaptations, variations, alternatives and uses of the invention, including what is presently believed to be the best mode of carrying out the invention.

Numerous specific details are set forth to provide a thorough understanding of the overall structure, function, manufacture, and use of the versions as described in the specification and illustrated in the accompanying drawings. Well-known operations, components, and elements have not been described in detail so as not to obscure the versions described in the specification. The reader will understand that the versions described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a robotic platform manipulating the housing portion of the surgical instrument. The term “proximal” refers to the portion closest to the robotic platform and the term “distal” refers to the portion located away from the robotic platform. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Furthermore, the terms “about,” “approximately,” “substantially,” and the like as used herein in connection with any numerical values, ranges of values, and/or geometric/positional quantifications are intended to encompass the exact value(s) or quantification(s) referenced as well as a suitable tolerance that enables the referenced feature or combination of features to function for the intended purpose described herein. For example, “substantially parallel” encompasses nominally parallel structures, and “substantially equal” values encompass nominally equal values.

Furthermore, the use of “couple”, “coupled”, or similar phrases should not be construed as being limited to a certain number of components or a particular order of components unless the context clearly dictates otherwise.

### I. Overview of Illustrative Surgical Instrument

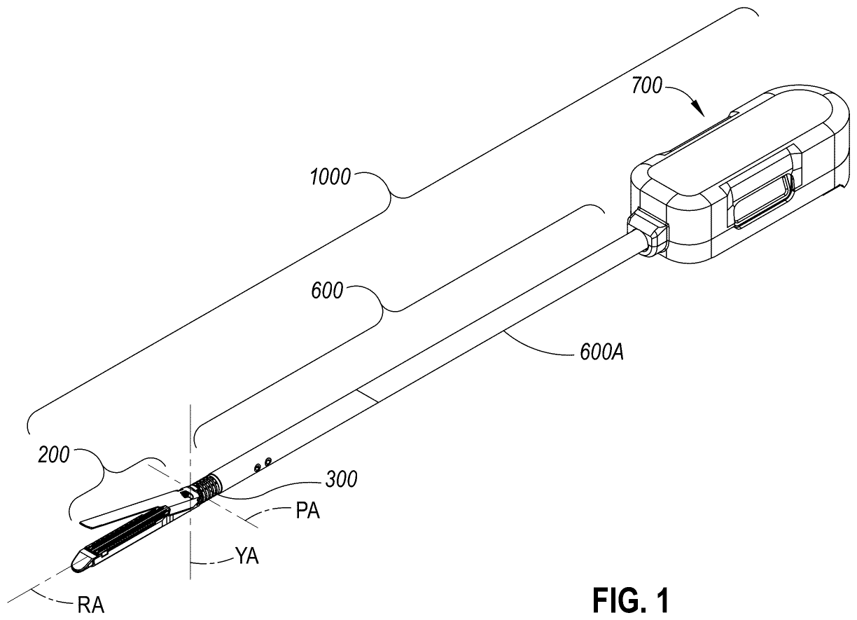

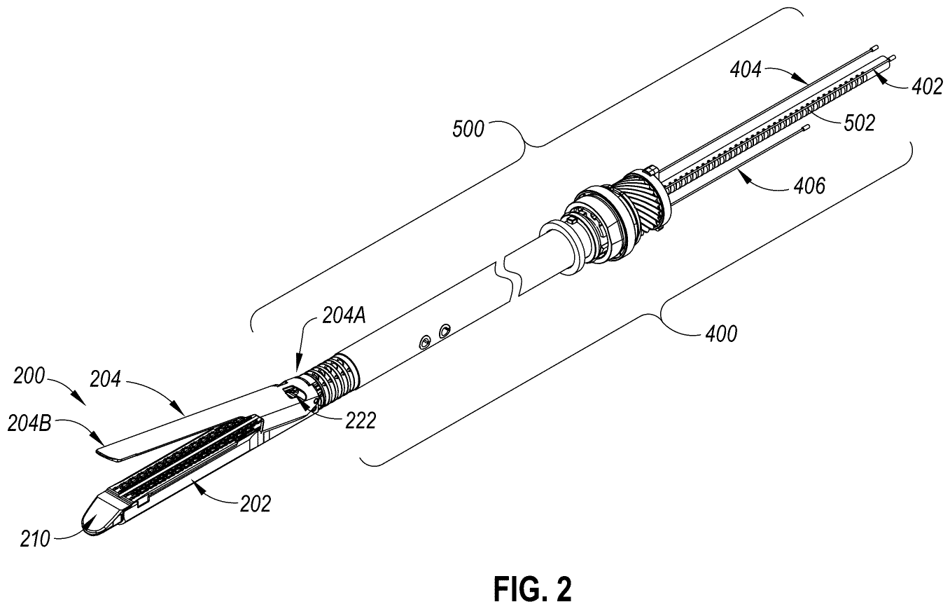

FIGS. 1-2 show an illustrative surgical instrument 1000 that is configured to grasp, clamp, incise, and seal patient tissue with staples. The surgical instrument 1000 comprises an end effector 200, an articulation joint 300 (also referred to as a “continuum joint”), an articulation drive subsystem 400 configured to articulate the end effector 200 via the articulation joint 300, a knife firing subsystem 500 configured to actuate the end effector 200 between various positions (e.g., an open position, a grasping position, and a clamping position) and to incise and staple patient tissue, a roll subsystem 600 configured to rotate the end effector 200 about a roll axis RA, and a housing 700.

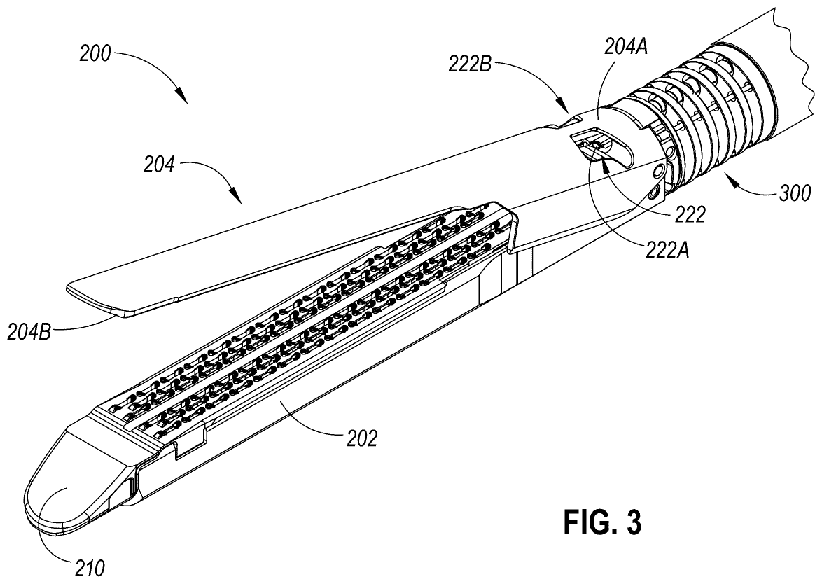

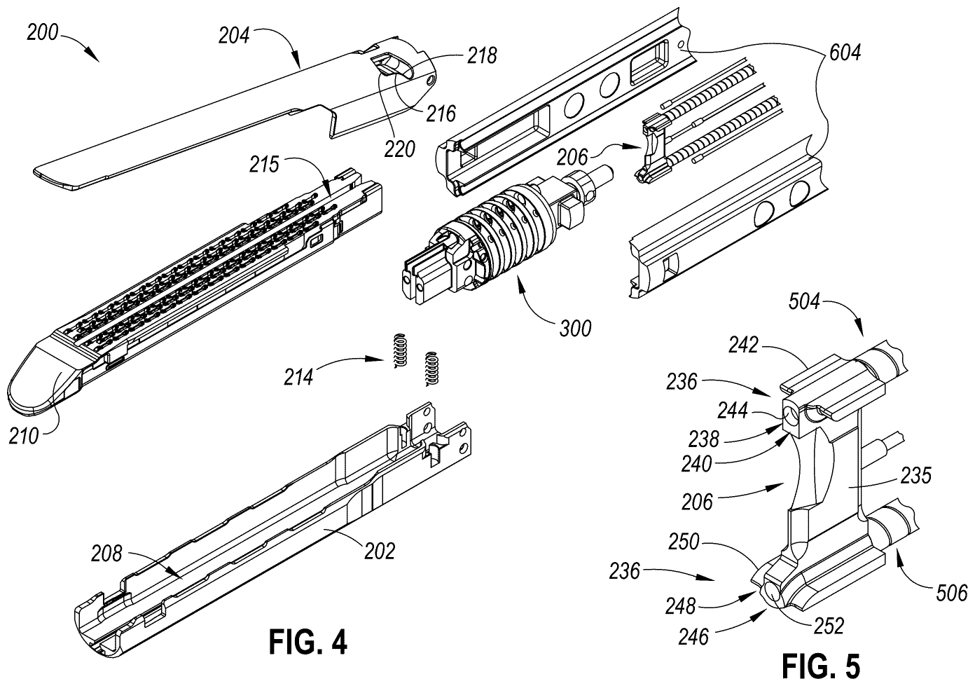

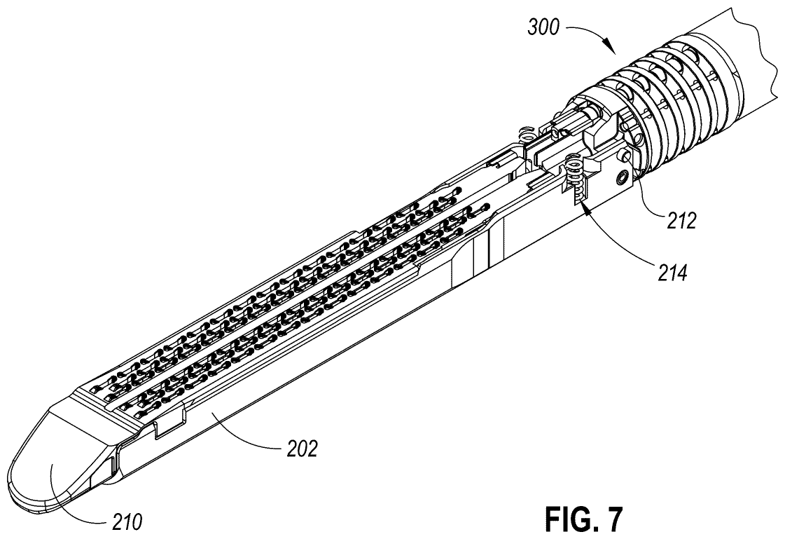

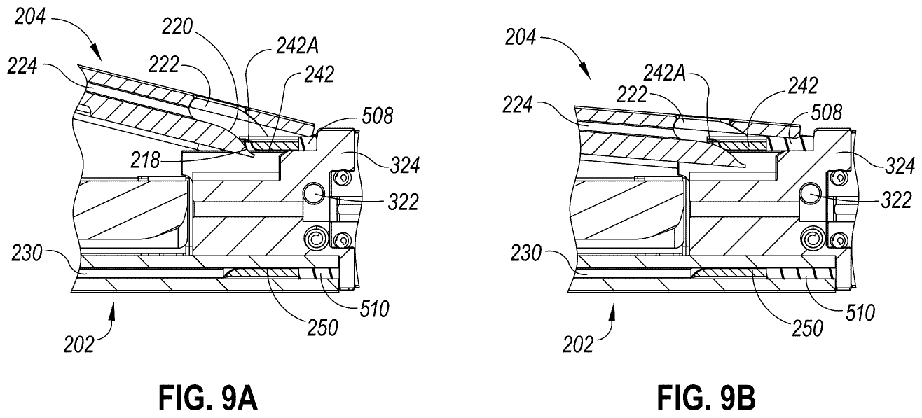

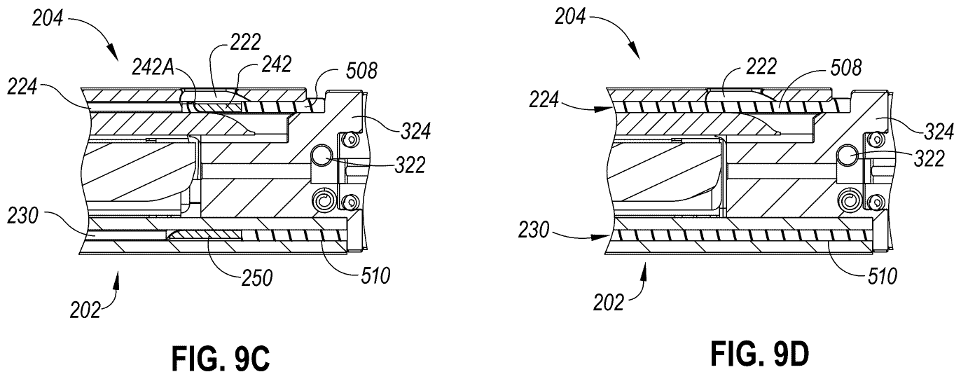

As shown best in FIGS. 3-4, the end effector 200 comprises a first jaw 202 (also known as a “cartidge jaw” or a “channel”) and a second jaw 204 (also known as an “anvil jaw” or just “anvil”) movable relative to the cartridge jaw 202 between an open position and a closed position. The cartridge jaw 202 and anvil 204 may be elongated in form. The cartridge jaw 202 defines an elongated channel 208 for receiving a staple cartridge 210 (also known as a “reload”). The anvil 204 has a proximal end 204A, a distal end 204B, and a ramp surface 216 defined at the proximal end 204A, which is described in greater detail below with respect to FIGS. 4 and 9A-9D. The cartridge jaw 202 and anvil 204 are pivotally coupled via a pivot pin 212 that extends through the cartridge jaw 202 and the anvil 204. As seen in FIG. 7, one or more biasing springs 214 extend between the cartridge jaw 202 and anvil 204 to bias the anvil 204 to the open position.

The ramp surface 216 may be visible via a kidney bean-shaped opening 222 (which may be formed as part of the manufacturing process to make the ramp surface 216) that has a first lateral end 222A and a second lateral end 222B. In other words, the kidney bean-shaped opening may be open at its lateral ends 222A, 222B (FIG. 3). As seen in FIG. 4 the ramp surface 216 forms a lower surface of the kidney bean-shaped opening 222. The ramp surface 216 can be arcuately shaped. For example, as shown particularly in FIGS. 4 and 9A-9D, it may be upwardly sloped at a first angle 218 and arcuately taper, in a distal direction, to a substantially horizontal second angle 220.

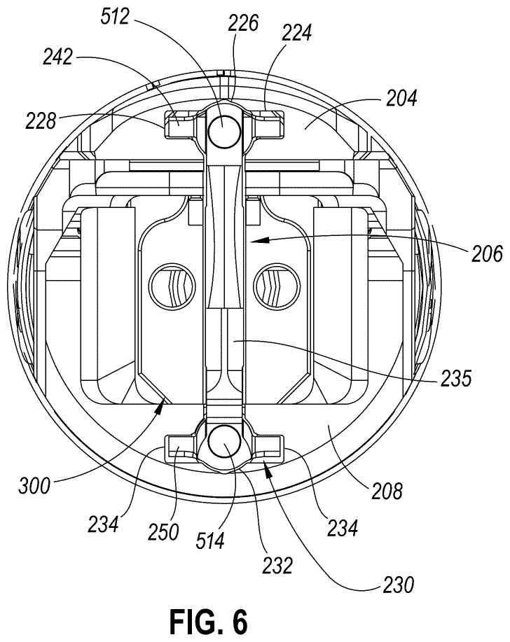

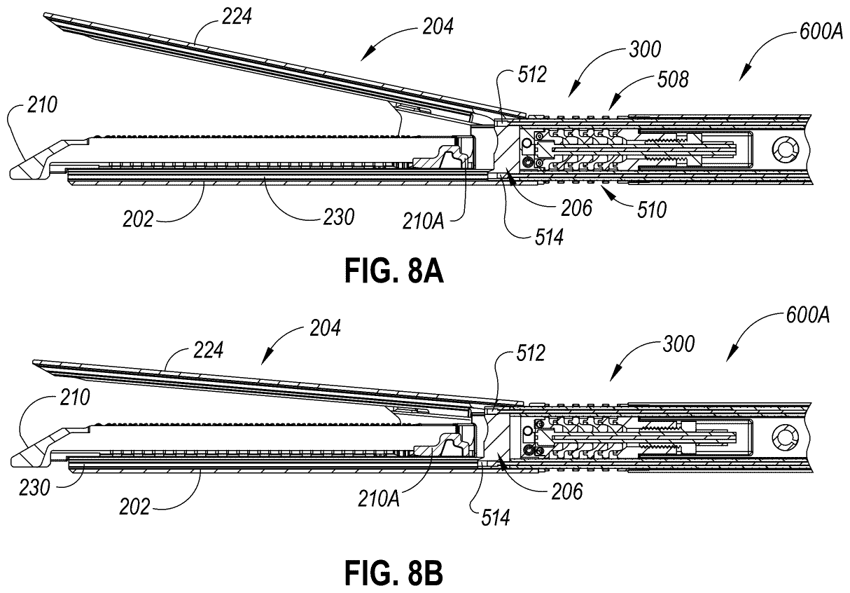

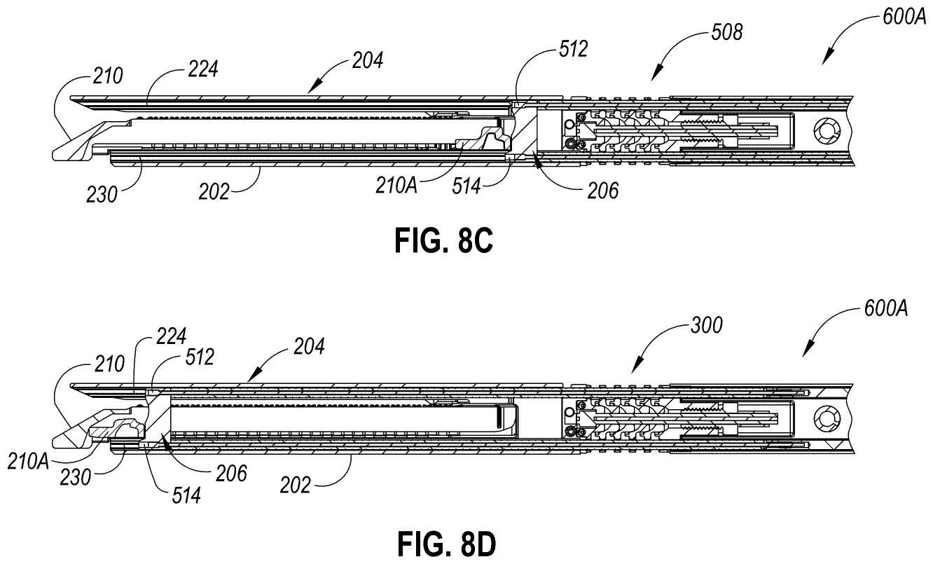

The anvil 204 further defines a longitudinally extending upper knife channel 224 (see FIG. 8A, etc.). As shown particularly in FIG. 6, the upper knife channel 224 includes a centrally disposed cylindrical upper knife channel portion 226 and at least one lateral upper knife channel wing 228 that extends away from the upper knife channel portion 226. While the term ‘cylindrical’ is used, the channel portion 226 need not resemble a perfect cylinder.

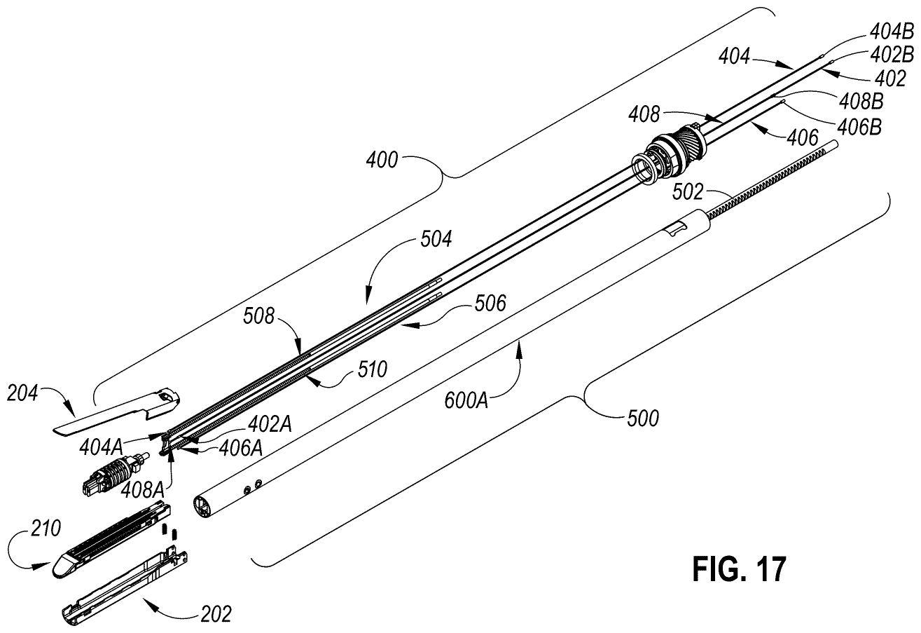

As shown in FIGS. 2 and 17, the surgical instrument 1000 further comprises a knife firing subsystem 500 operable to close the anvil 204 during a closure stroke. After the end effector 200 is closed, the knife firing subsystem 500 is operable to incise and staple, with staples from the staple cartridge 210, the patient tissue captured between the staple cartridge 210 (which is retained by the cartridge jaw 202) and anvil 204 during a firing stroke.

As shown best in FIGS. 4-6, the knife firing subsystem 500, explained further below in greater detail, includes a knife 206 having a knife sled 236. The knife sled 236 is the non-cutting element of the knife 206 and functions as a firing driver by driving cartridge sled 210A distally through a firing stroke, as described below. In some instances, knife sled 236 may be referred to as an I-beam. The knife sled 236 includes an upper knife tab 238, a lower knife tab 246, and a vertical column 235 coupling and extending between upper knife tab 238 and lower knife tab 246. The upper knife tab 238 includes a centrally disposed cylindrical upper knife tab portion 240 and at least one upper knife tab lateral wing 242 that extends away from the upper knife tab portion 240. While the term ‘cylindrical’ is used, the tab portion need not resemble a perfect cylinder.

The upper knife tab 238 may include a pair of lateral wings 242 configured to slidably ride in the upper knife channel 224 to move the anvil 204 between the open position, the grasping position, and the clamping position. Accordingly, the end effector 200 employs “knife-based closure” in which closure of the anvil 204 relative to the channel 208 is driven by distal advancement of the knife 206. Each lateral wing 242 may include a ramped surface 242A that engages the anvil ramp surface 216. The upper knife tab portion 240 defines an upper knife tab opening 244 that is configured to receive a barrel crimp coupled to a center cable 512, which is described in greater detail below. The lower knife tab 246 includes a centrally disposed cylindrical lower knife tab portion 248 and at least one lower knife tab lateral wing 250 that extends away from the lower knife tab portion 248. While the term ‘cylindrical’ is used, the lower knife tab portion 248 need not resemble a perfect cylinder. In some versions, the lower knife tab 246 includes a pair of lateral wings 250. The lower knife tab portion 248 defines a lower knife tab opening 252 that is configured to receive a barrel crimp coupled to a center cable 514, as described in greater detail below.

The staple cartridge 210 may be generally constructed and operable in accordance with the teachings of U.S. patent application Ser. No. 18/588,684, entitled “Methods of Surgical Stapling,” filed on Feb. 27, 2024, the disclosure of which is incorporated by reference herein in its entirety. In use, the end effector 200 is positioned relative to patient tissuc such that the staple cartridge 210 is disposed on a first side of the tissue and the anvil 204 is positioned on an opposed second side of the tissue. The anvil 204 is then approximated toward the staple cartridge 210 to compress and clamp the tissue against the deck of the staple cartridge 210. Thereafter, the surgical instrument 1000 is fired so that the knife 206 advances distally through the staple cartridge 210 to both cut the clamped tissue and simultaneously actuate staple drivers housed within the staple cartridge 210 to drive an array of staples into the clamped tissue on either side of the cut line. Staple cartridge 210 defines an elongate knife channel 215 dimensioned to receive a portion of vertical column 235 in order to accommodate advancement of knife 206 through staple cartridge 210. A portion of cartridge sled 210A is slidably housed within elongate knife channel 215 such that vertical column 235 drives cartridge sled 210A distally as knife 206 advances distally in accordance with the description herein (see FIGS. 8C-8D). In some instances, cartridge sled 210A remains in the distal position (see FIG. 8D) relative to the rest of staple cartridge 210, even after knife 206 is retracted proximally after firing staple cartridge 210 in accordance with the description herein.

As mentioned above, cartridge jaw 202 defines an elongated channel 208 for receiving staple cartridge 210. Additionally, cartridge jaw 202 also defines a lower knife channel 230 (see FIGS. 4, 6, and 8A-9D) dimensioned to slidably receive lower knife tab 246. Referring to FIG. 6, the lower knife channel 230 includes a centrally disposed cylindrical lower knife channel portion 232 and at least one lateral lower knife channel wing 234 that extends away from the lower knife channel portion 232. Cylindrical lower knife channel portion 232 is in communication with elongated channel 208 such that when staple cartridge 210 is suitably coupled to cartridge jaw 202, elongate knife channel 215 of staple cartridge 210 and centrally disposed cylindrical lower knife channel portion 232 are aligned to accommodate actuation of knife sled 236 within both channels 215, 230. Lateral lower knife channel wings 234 are dimensioned to slidably house a respective lower knife tab lateral wing 250. Lower knife tab lateral wings 250 are configured to slidably contact lateral lower knife channel wings 234 as knife 206 is advanced in accordance with the description herein. Contact between lower knife tab lateral wings 250 and lateral lower knife channel wings 234 cooperatively assists lateral wings 242 and upper knife channel 224 to close anvil 204 relative to channel 208 in accordance with the description herein. While the term ‘cylindrical’ is used, the channel portion 232 need not resemble a perfect cylinder. Other arrangements of staple cavities and staples may be possible. For example, in some versions, a lower knife channel 230 can be defined in the cartridge jaw 202.

Further to the above, the knife sled 236 is moved distally and proximally by a firing rod 502. The firing rod 502 is configured to apply an indirect force to the knife sled 236, via push coils 508, 510 that directly engage the knife sled 236 (discussed in greater detail below), and push the knife sled 236 toward the distal end of the end effector 200 through a firing stroke. Bendable rods, such as nitinol rods, may act as an alternative to push coils 508, 510. As the firing rod 502 is advanced distally, knife sled 236 rides in the lower knife channel 230 and the upper knife channel 224. At the onset of travel, the upper knife tab 238 rides along the anvil ramp surface 216. Specifically, as particularly seen in the sequence of FIGS. 8A-8D and 9A-9D, movement of the knife sled 236 distally causes the upper knife tab ramped surface 242A to slide along the anvil ramp surface 216. This movement first urges the anvil 204 closed to a position (e.g., FIGS. 8B and 9B) where a compressive force is applied to the tissue sufficient to grasp it (referred to as the grasping position). Continued movement of the knife sled 236 up the ramp surface 216 (e.g., see FIGS. 8C and 9C) results in a compressive force being applied to the tissue (referred to as the clamping position). As the anvil ramp surface 216 transitions to its substantially horizontally angled surface 220 (e.g., see FIGS. 8D and 9D), the upper knife tab 238 can slide within the upper knife channel 224 to drive the stapling and transection of the tissue.

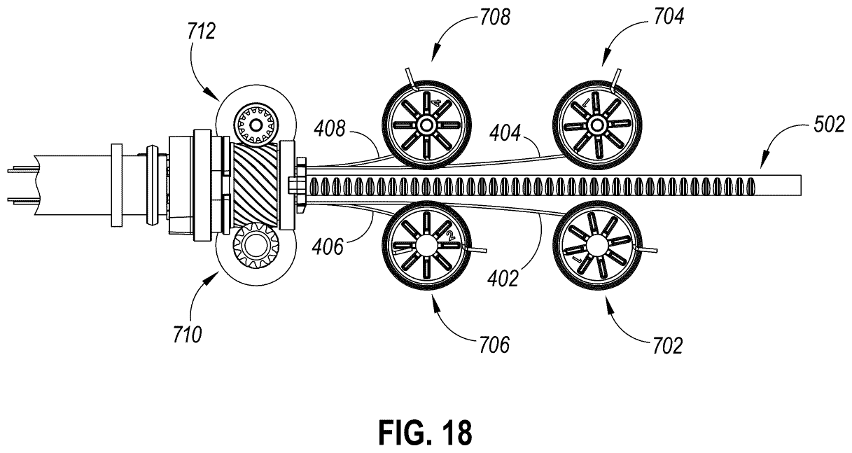

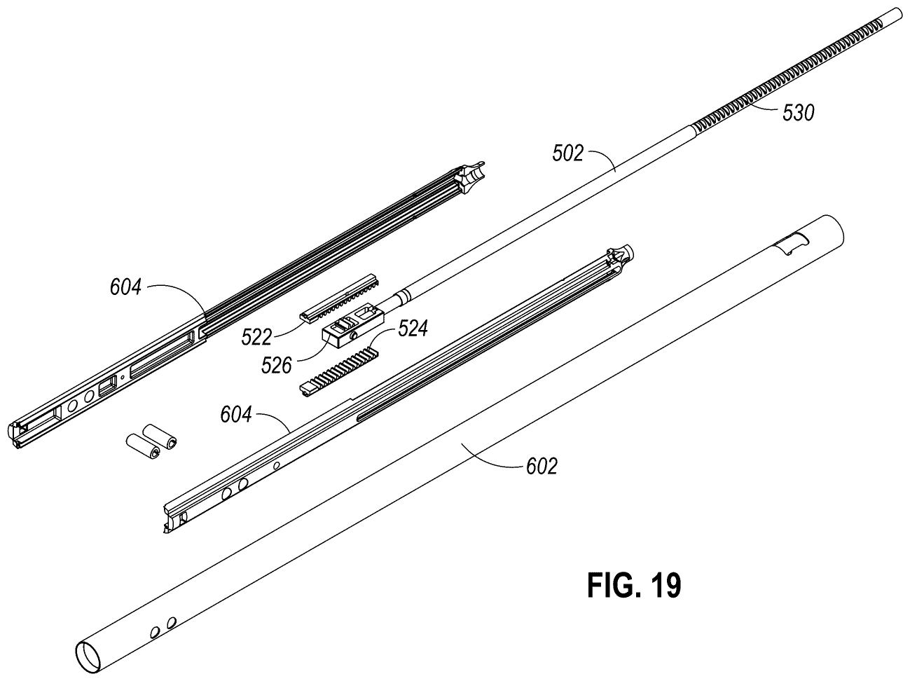

As shown in FIG. 1, the surgical instrument 1000 further comprises a body exemplified as a housing 700 configured to engage a robotic platform (not shown). In other versions, the body may be configured as a handle configured to be gripped and manipulated by a clinician. As best shown in FIGS. 1 and 19, a shaft assembly 600A extends distally from the housing 700 and includes a rotatable outer shaft 602 and an inner shaft 604 arranged in two clamshell halves, with the outer shaft 602 being rotatably mounted to the housing 700 about a rotation joint (not shown), which may include one or more bearings. The inner shaft 604 is rotationally fixed to the outer shaft 602 and is configured such that articulation cables 402, 404, 406, 408 can be partially wound therearound without becoming tangled. As shown in FIG. 18, the housing 700 may house (1) a firing puck assembly 712 as part of the knife firing subsystem 500 operable to close the end effector 200, fire staples, and transect tissue, (2) a set of articulation puck assemblies 702, 704, 706, 708 as part of the articulation subsystem 400 operable to articulate the end effector 200 relative to the shaft assembly 600A, and (3) a shaft roll puck assembly 710 as part of the roll subsystem 600 configured to roll the outer shaft 602.

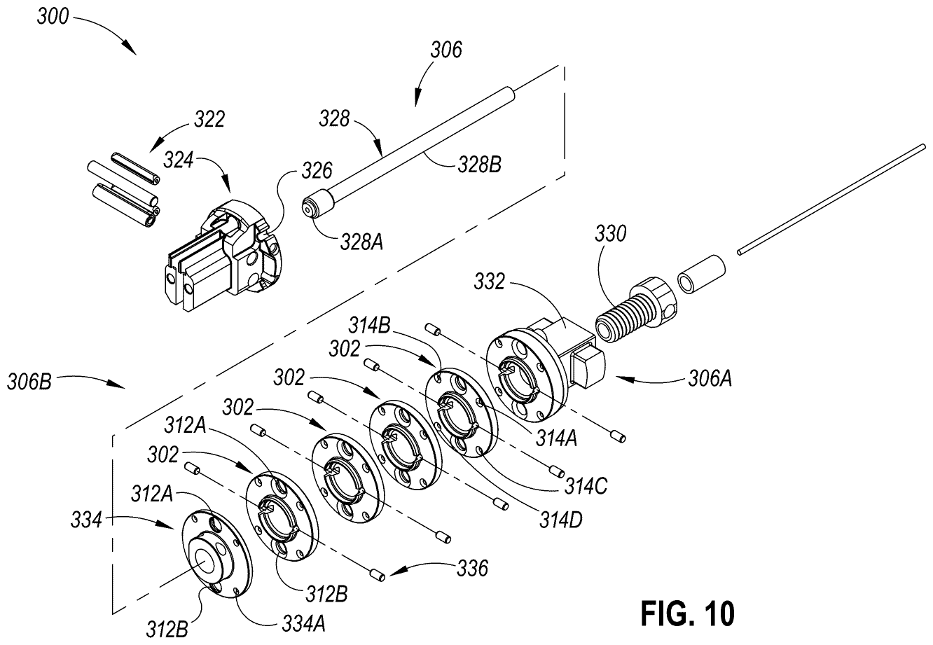

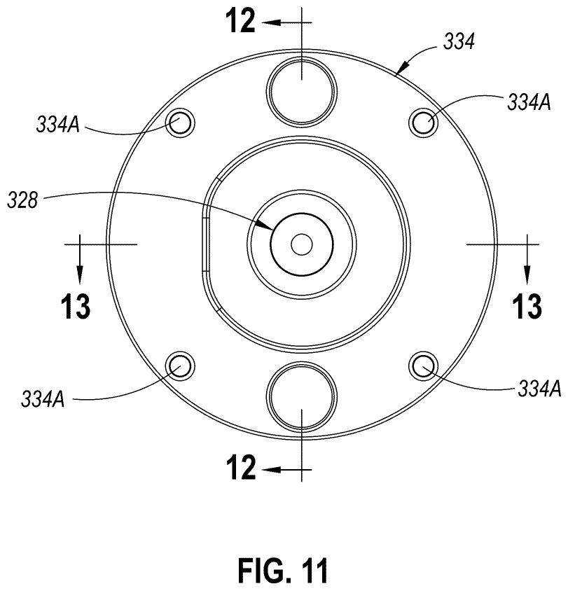

Referring to FIGS. 10-13, the articulation joint 300 comprises an array of joint discs 302 arranged longitudinally, and a center beam assembly 306 that cooperates with the joint discs 302 to provide articulation of the end effector 200 with at least two degrees of freedom (e.g., yaw and pitch), as described further below. Each joint disc 302 includes a central opening 304 that is configured to align coaxially with the central opening 304 of the other joint discs when the articulation joint 300 is in a straight, non-articulated state. The center beam assembly 306 extends longitudinally through the central openings 304 of joint discs 302 and applies a compressive axial force to the array of joints discs 302 to couple the joint discs 302 with one another. The joint discs 302 are nestably stacked with one another along the center beam assembly 306 such that longitudinally adjacent joint discs 302 movably interface with one another.

As seen in FIGS. 9A-10, a distal end 306B of the center beam assembly 306 includes a distal retainer 324 that couples the distal end of the articulation joint 300 with a proximal end of the cartridge jaw 202 via one or more fasteners 322, thereby mechanically grounding and retaining the cartridge jaw 202 and thus the end effector 200 relative to the articulation joint 300. The distal retainer 324 includes a plurality of clearance pockets 326 that receive distal ends of articulation cables 402, 404, 406, 408. The distal end 306B further includes a distal retention disc 334 that defines a plurality of cable retention openings 334A. A proximal end 306A of the center beam assembly 306 includes a proximal retainer 332 that couples the proximal end of the articulation joint 300 with a distal end of the shaft assembly 600A.

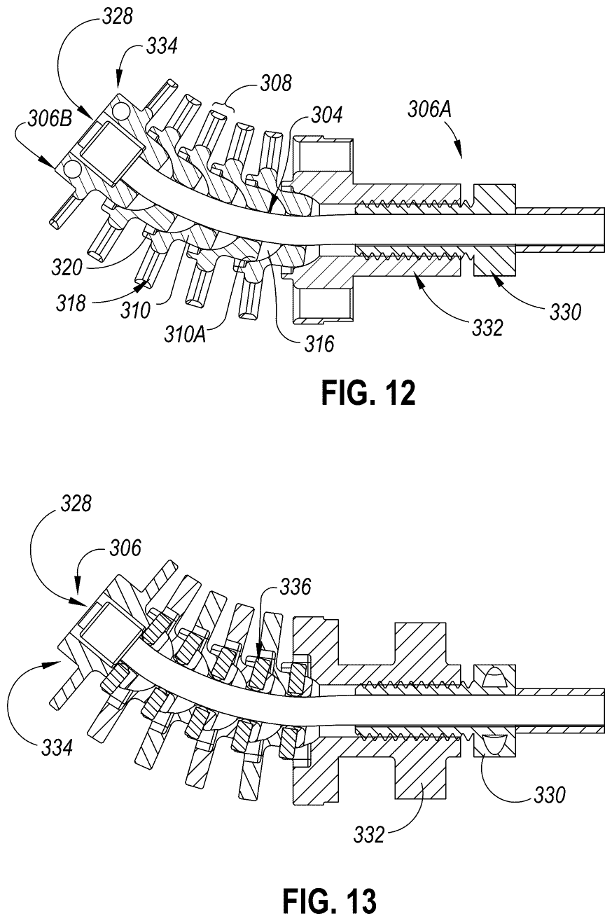

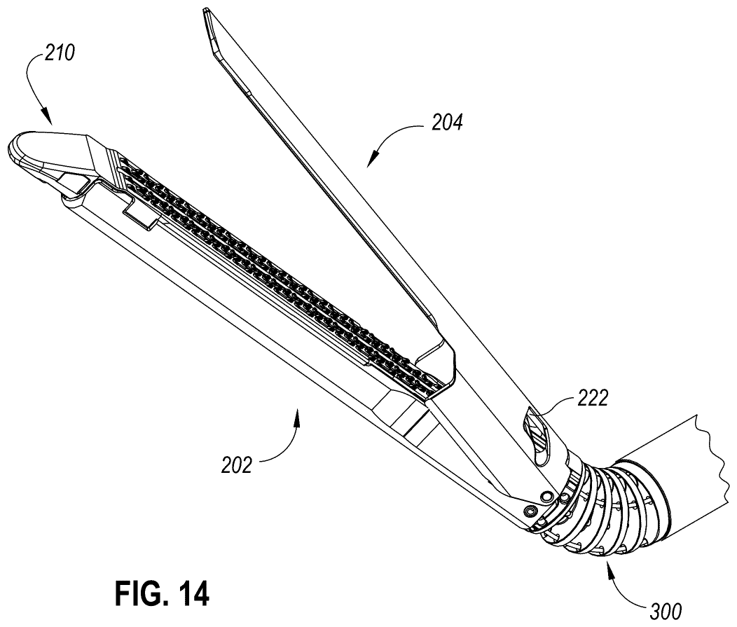

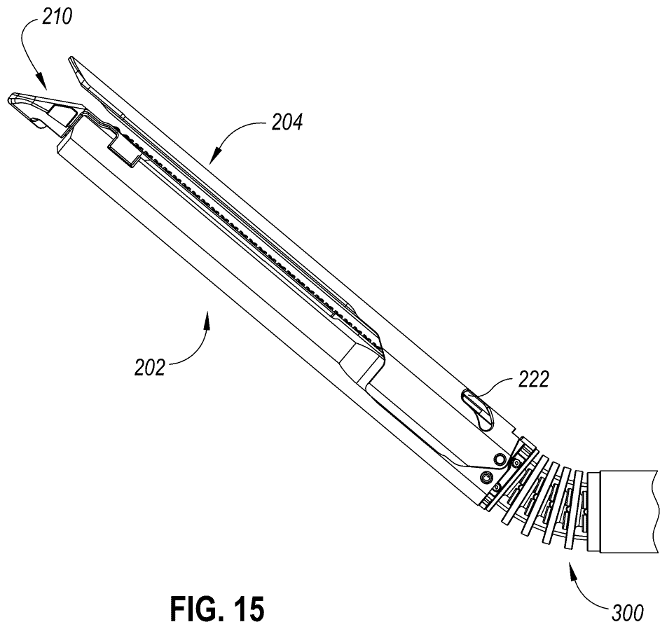

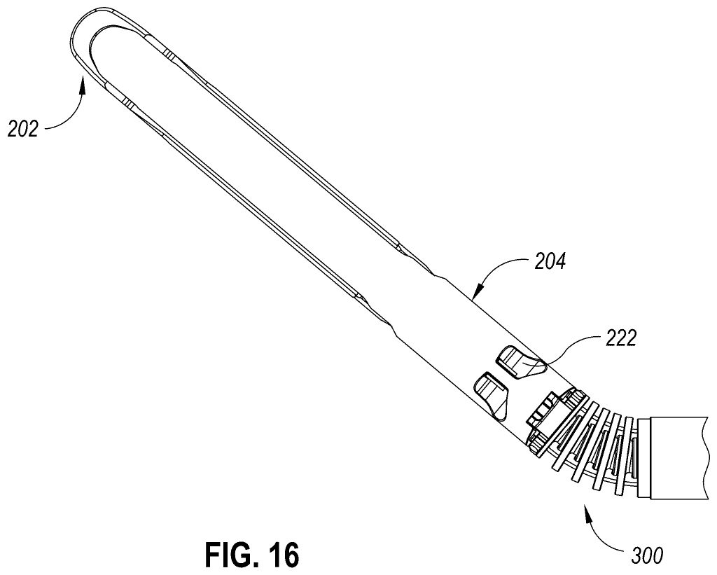

As shown particularly in FIGS. 10, 12, and 13, each joint disc 302 includes an articulation socket 308, an articulation pin 310 protruding outwardly from the articulation socket 308, a first push coil opening 312A defined through the articulation socket 308 and configured to receive a first push coil 508 therethrough, a second push coil opening 312B defined through the articulation socket 308 and configured to receive a second push coil 510 therethrough, and a plurality of articulation cable openings 314A-314D (e.g., a first articulation cable opening 314A, a second articulation cable opening 314B, a third articulation cable opening 314C, and a fourth articulation cable opening 314D) defined through the articulation socket 308 and configured to receive a respective articulation cable 402, 404, 406, 408 (e.g., a first articulation cable 402, a second articulation cable 404, a third articulation cable 406, and a fourth articulation cable 408) therethrough, and discussed in greater detail below. As shown in FIGS. 12 and 13, the central opening 304 is defined in the articulation pin 310 of each joint disc 302. In some versions, three articulation cable openings 314A, 314B, 314C are provided to correspond to three articulation cables 402, 404, 406, while in other versions, four articulation cable openings 314A, 314B, 314C, 314D are provided to correspond to four articulation cables 402, 404, 406, 408.

Each joint disc 302 further includes a rounded articulation pin proximal end 310A and a semi-spherical pin-receiving opening 316 defined in the articulation socket 308. As shown particularly in FIGS. 12 and 13, each rounded articulation pin proximal end 310A pivotally engages in an adjacent pin-receiving opening 316 of an adjacent joint disc 302, with the exception of a proximal-most end 310A that engages with the proximal retainer 332. The articulation pin proximal end 310A and pin-receiving opening 316 interface functions in a similar manner as a swivel bearing. Moreover, the articulation socket 308 includes a socket disc 318 and a pin retention socket 320. A pair of pins 336 are used to provide rotational coupling about a primary axis of the shaft assembly 600A from one disc 302 to the next. In other words, the pins constrain a rotational degree of freedom between adjacent joint discs 302 about the roll axis RA of the instrument 1000. In alternative versions, this feature can be integral to the joint disc 302.

The center beam assembly 306 further includes a center beam 328 that extends longitudinally through the central openings 304 of the joint discs 302. The center beam 328 includes a nitinol core 328A and a stainless-steel collar 328B wound over the nitinol core 328A that allows the center beam 328 to resiliently flex during deflection of the articulation joint 300. The wound stainless-steel collar 328B may have clockwise braiding and counterclockwise braiding to prevent unwinding thereof. The center beam assembly further includes a jack screw 330 that is threadably coupled with the proximal retainer 332 to adjust an axial compression force exerted by the center beam 328 on the array of joint discs 302, thereby enabling adjustment of a pre-load of the articulation joint 300.

The above-described articulation joint 300 forms a portion of the cable articulation subsystem 400 which allows for precise 360-degree movement of the end effector 200 about the articulation joint 300 with at least two degrees of freedom. In some versions, and as dictated by the roll subsystem 600 as well as a need to limit the amount of wrap of the articulation cables 402, 404, 406, 408, the articulation joint 300 is permitted about 320 degrees of roll within the overall system. The cable articulation subsystem 400 also includes a plurality of articulation cables 402, 404, 406, 408 each having a distal end 402A, 404A, 406A, 408A, coupled to the distal end 306B of the center beam assembly 306, and a proximal end 402B, 404B, 406B, 408B. More specifically, cach distal end 402A, 404A, 406A, 408A can include a crimp that engages a cable retention opening 334A of the distal retention disc 334 to maintain its positioning. Each articulation cable is discretely manipulable to cause rotation of the articulation joint 300 and end effector 200 about at least one of a pitch axis PA and a yaw axis YA.

In some versions, three articulation cables may be provided rather than the four cables 402, 404, 406, 408 depicted herein. However, four articulation cables 402, 404, 406, 408 circumferentially spaced approximately ninety degrees from one another (as shown) provide load splitting. Additionally, in alternative versions, three and fourth articulation cable configurations may be spaced non-symmetrically relative to one another.

The shaft assembly 600A and housing 700 also form portions of the cable articulation subsystem 400. More specifically, each articulation cable 402, 404, 406, 408 extends from the articulation joint 300 and through the shaft assembly 600A to the housing 700. The proximal end 402B, 404B, 406B, 408B of cach articulation cable (402, 404, 406) is movably mounted in the housing 700 which causes the above-mentioned rotation of the articulation joint 300 and end effector 200. The housing 700 includes articulation puck assemblies 702, 704, 706, 708 with rotatable capstans (not shown) about which corresponding proximal ends 402B, 404B, 406B, 408B of the articulation cables 402, 404, 406, 408 are windably mounted.

The articulation cables 402, 404, 406, 408 are routed through the shaft assembly 600A such that they are disposed between the outer shaft 602 and the inner shaft 604, with the articulation cables 402, 404, 406, 408 being able to partially wind therearound without becoming tangled. The inner shaft 604 also prevents the articulation cables 402, 404, 406, 408 from interfering with other components running down the center of the instrument 1000 (through the inner shaft 604).

The articulation cables 402, 404, 406, 408 are routed and coupled to the end effector 200 via the articulation joint 300 such that movement thereof in a proximal direction (via winding about the capstans of the housing 700) causes the end effector 200 to articulate in a predetermined manner via the articulation joint 300. For example, actuation of the first articulation cable 402 in the proximal direction causes articulation of the end effector 200 upwards and to the left, actuation of the second articulation cable 404 in the proximal direction causes rotation of the end effector 200 upwards and to the right, actuation of the third articulation cable 406 in the proximal direction causes rotation of the end effector 200 downwards and to the left, and actuation of the fourth articulation cable 408 in the proximal direction causes rotation of the end effector 200 downwards and to the right. Similarly, movement of two articulation cables simultaneously will result in blended articulation of the end effector 200. As will be appreciated by those skilled in the art, this configuration provides for the above-mentioned precise 360-degree articulation of the end effector 200 via the articulation joint 300 with at least two degrees of freedom and about 320 degrees of roll.

As shown throughout FIGS. 2, 4, 5, 8A-8D, 9A-9D, 17 and 19, the knife firing subsystem 500 includes the aforementioned knife 206, the aforementioned knife sled 236, a firing rod 502 that drives the knife 206 and/or knife sled 236, a first push rod 504, and a second push rod 506. The firing rod 502 includes a firing rod rack 530 and is driven by a firing puck assembly 712 of the housing 700. The first push rod 504 has a first push rod distal end 504A coupled to the knife sled 236 and a first push rod proximal end 504B coupled to the firing rod 502. Similarly, the second push rod has a second push rod distal end 506A coupled to the knife sled 236 and a second push rod proximal end 506B coupled to the firing rod 502. The distal ends 504A, 506A are coupled to respective upper and lower portions of the knife sled 236 (e.g., the upper knife tab 238 and the lower knife tab 246), which enables the knife 206 to be pushed evenly at its ends. In some versions, the proximal ends 504B, 506B of the push rods 504, 506 are coupled to the firing rod 502 via a differential 520.

The knife firing subsystem 500 is configured in a manner to enable articulation of the end effector 200 while still enabling proper functionality of the knife 206. To that end, the first push rod 504 includes a first flexible section in the form of a first push coil 508 and the second push rod 506 comprises a second flexible section in the form of a second push coil 510. The push coils 508, 510 route through the articulation joint 300 via the respective push coil openings 312A, 312B, and the push rods 504, 506 engage the respective tab openings 244, 252 in the knife sled 236. A first center cable 512 extends through the first push coil 508 to engage the knife sled 236 via a barrel crimp, and a second center cable 514 extends through the second push coil 510 to engage the knife sled 236 via a barrel crimp. The push coils 508, 510 provide the push rods 504, 506 sufficient stability to deliver an axial firing force to the knife 206, while not being too stiff that would prevent articulation at the joint 300. The cables 512, 514, which are engaged with the knife sled 236 as discussed above (see, e.g., FIG. 8A), prevent the push coils 508, 510 from stretching and/or clongating and serve as retraction cables when the rods 504, 506 are retracted towards the proximal end of the surgical instrument 1000. The entirety of each push rod 504, 506 does not extend through the articulation joint 300, and therefore does not need to be flexible. Accordingly, a proximal section of each push rod 504, 506 can be less flexible than the push coils 508, 510.

### II. Illustrative Alternative Articulation Joint With Rigid Links

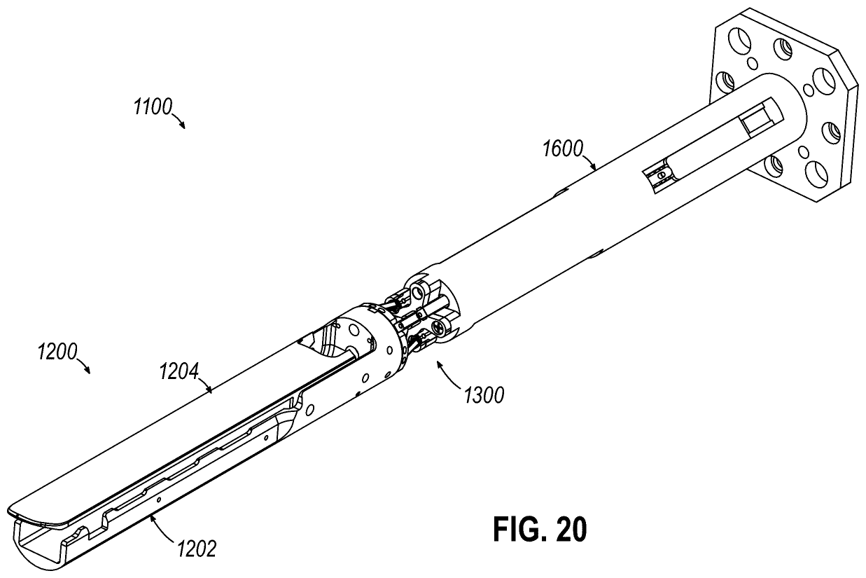

It may be desirable to alternatively configure the articulation joint 300 of the surgical instrument 1000 to replace the pull-only articulation cables 402, 404, 404, 408 with an assembly of rigid links that longitudinally span the join and can be pushed and pulled to articulate the end effector about each of a yaw axis and a pitch axis. An articulation joint using rigid links rather than cables may be beneficial to allow for increased articulation at the joint. An articulation joint using linkages may also be beneficial by limiting the number of motors needed to achieve multi-plane articulation. Rigid links may be less susceptible than traditional articulation cables to unwanted stretching, and therefore such an articulation joint may minimize unwanted dearticulation of the end effector while resisting external loads applied to the end effector during a firing stroke on patient tissue. Dearticulation may be defined as a change in the articulation joint pose that is not a result of a control input such as cable displacement. Rather, it will be understood that dearticulation is the result of an external force or moment that overwhelms the articulation system causing it to change pose. FIGS. 20-26B show illustrative variations of such an articulation joint (also referred to herein as an articulation assembly).

As shown in FIG. 20, surgical instrument 1100 includes an end effector 1200 coupled to an articulation joint 1300 coupled to a shaft 1600. Surgical instrument 1100 may be substantially similar to surgical instrument 1000 and may function in substantially the same way, except as otherwise described below. As will be described, end effector 1200 includes a cartridge jaw 1202 and an anvil jaw 1204 that articulate relative to shaft 1600 via articulation joint 1300. Surgical instrument 1100 may include two articulation inputs, yaw and pitch, to articulate end effector 1200 into various poses. Collectively, these inputs to articulation joint 1300 allow end effector 1200 to articulate relative to shaft 1600 in 360 degrees worth of poses while also rotationally fixing end effector 1200 to shaft 1600.

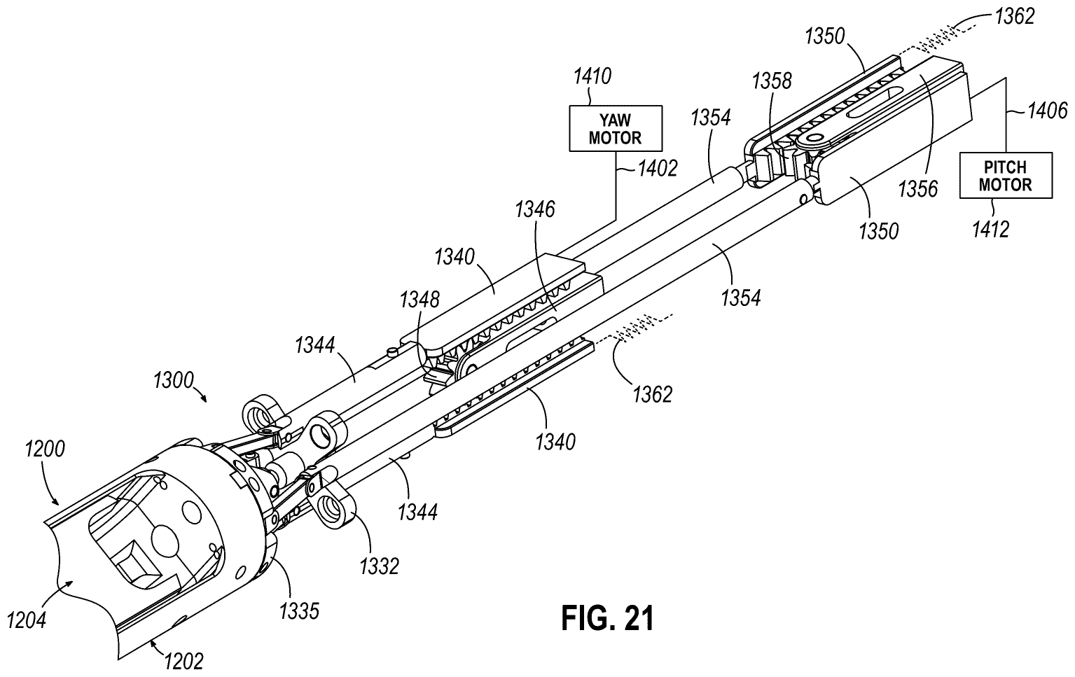

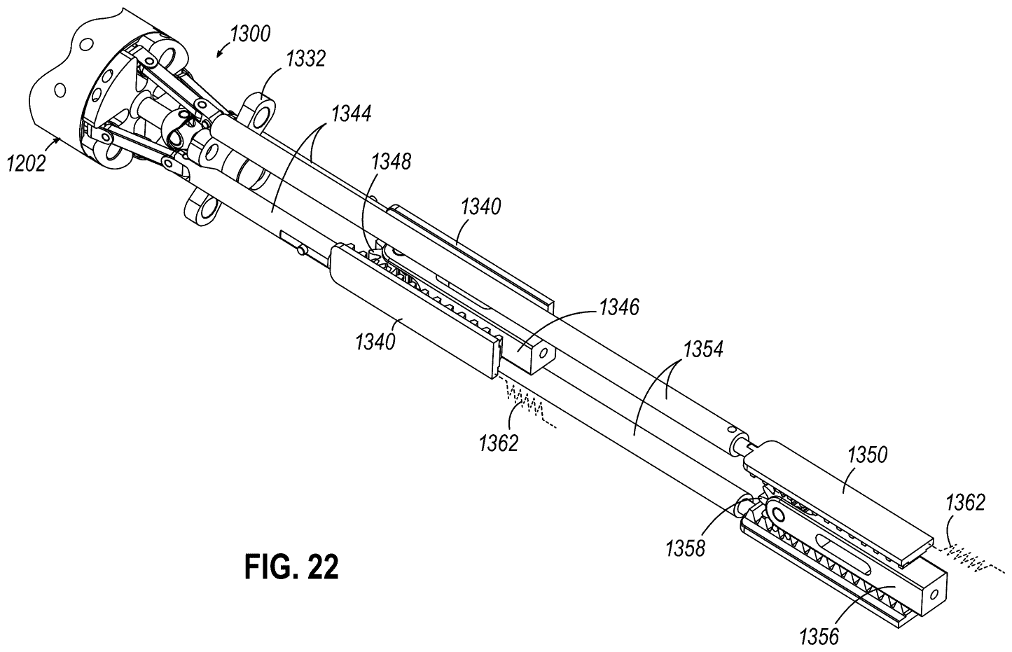

FIGS. 21-22 show surgical instrument 1100 absent shaft 1600 to better show the internal components of the system. End effector 1200 is coupled to articulation joint 1300 via a distal retainer 1335 while shaft 1600 is coupled to articulation joint 1300 via a proximal retainer 1332. As discussed above, articulation of surgical instrument 1100 is driven via two inputs, a pitch input and a yaw input. Each of the pitch and yaw assemblies are substantially similar to each other although they are circumferentially offset by 90 degrees from each other to thereby accomplish the respective pitch or yaw articulation.

Discussing the yaw drive assembly from distal to proximal, articulation joint 1300 is coupled to first and second yaw articulation beams 1344 that are laterally opposed about a longitudinal axis of shaft 1600. Each yaw articulation beam 1344 is then coupled to a respective yaw rack gear 1340. Each of the yaw articulation beams 1344 and the yaw rack gears 1340 translates along a path parallel to the longitudinal axis of shaft 1600 to thereby drive yaw articulation of end effector 1200. Yaw articulation beams 1344 are diametrically opposed and spaced apart from the other about the longitudinal axis. As discussed below, pitch articulation beams 1354 of the pitch drive assembly are also diametrically opposed and circumferentially offset from yaw articulation beams 1344 such that yaw and pitch articulation beams 1344, 1354 are circumferentially spaced apart approximately 90 degrees from each other in an alternating arrangement. Each of the yaw rack gears 1340 is meshed with yaw pinion gear 1348, which is coupled to a yaw carrier 1346 interposed between yaw rack gears 1340. As will be described in greater detail, yaw carrier 1346 may be fixed relative to shaft 1600 to thereby prevent yaw carrier 1346 from translating longitudinally or may be floating relative to shaft 1600 to thereby allow yaw carrier 1346 to translate longitudinally. A first yaw rack gear 1340 may then be operatively coupled with a yaw motor 1410 via a yaw drive coupling 1402, which may be a rigid coupling or a flexible coupling, such as a cable for example. Through this coupling 1402, yaw motor 1410 may actuate the coupled first yaw rack gear 1340 distally. As the first yaw rack gear 1340 actuates distally, yaw pinion gear 1348 simultaneously rotates to thus drive the opposing second yaw rack gear 1340 in a proximal direction, thereby causing end effector 1200 to yaw in first yaw direction. The rotational direction of yaw motor 1410 may be reversed to drive the first rack gear 1340 proximally and the second rack gear 1340 distally to thereby cause end effector 1200 to yaw in a second yaw direction opposite the first yaw direction. A yaw spring 1362 may be operatively coupled to the second, non-driven yaw rack gear 1340 to resiliently bias the second, non-driven yaw rack gear 1340, and thus also the first, driven rack gear 1340, toward a non-actuated state to thereby bias the end effector 1200 toward a non-yawed orientation.

In some versions, each yaw rack gear 1340 may be coupled to respective opposing ends of an articulation cable 1402. In doing so, yaw motor 1410 may pull either yaw rack gear 1340 to thus actively drive end effector 1200 in either yaw direction. Spring 1362 may optionally be positioned between articulation cable 1402 and each yaw rack gear 1340 to thus allow for slack in articulation cable 1402.

As noted above, yaw motor 1410 may be coupled to rack gear 1340 via a coupling 1402 in the form of a rigid link or through direct drive. Said link or direct drive may be beneficial to thus avoid cable lengthening of a cable-driven configuration. In some such versions, pinion gear 1348 may be operatively coupled directly to yaw motor 1410 to drive rotation of pinion gear 1348 and thus articulate end effector 1200.

Pitch drive assembly may be substantially similar to the above-described yaw drive assembly except that it is circumferentially offset by 90 degrees to thus drive pitch rather than yaw of end effector 1200. Pitch drive assembly includes a diametrically opposed pair of pitch articulation beams 1354, a diametrically opposed pair of pitch rack gears 1350, a pitch pinion gear 1358, and a pitch carrier 1356 interposed between pitch rack gears 1350 and rotatably supporting pitch pinion gear 1358. One pitch rack gear 1350 may be coupled to a pitch spring 1362 while the other pitch rack gear 1350 may be coupled to a pitch drive coupling 1406 coupled to a pitch motor 1412. Pitch motor 1412 thus is capable of driving a pitch articulation of end effector 1200 via the pitch drive assembly and articulation joint 1300, while pitch spring 1362 is configured to resiliently bias end effector 1200 toward a non-pitched orientation. As shown, a proximal end of pitch drive assembly may be longitudinally offset from a proximal end of yaw drive assembly via a different length of a pitch articulation beams 1354; specifically, with pitch articulation beams 1354 being longer than yaw articulation beams 1344.

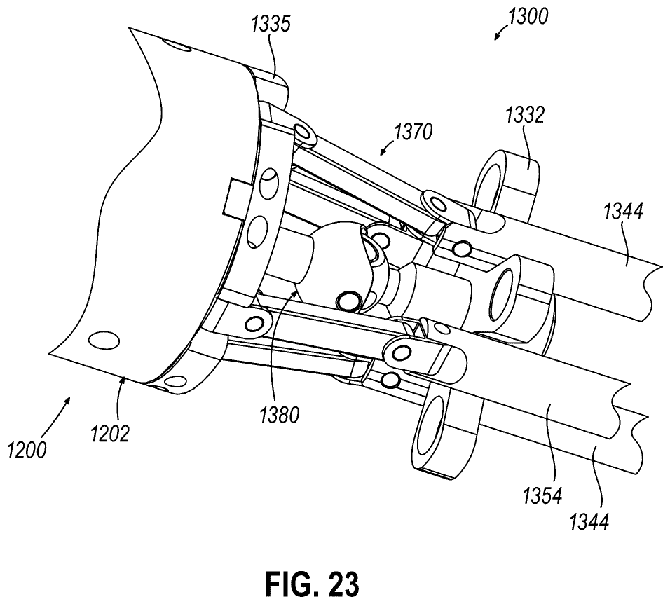

As shown in FIG. 23, articulation joint 1300 includes four link assemblies 1370 positioned circumferentially 90 degrees from each other about a constant velocity joint 1380. Each link assembly 1370 is coupled to a respective yaw articulation beam 1344 or pitch articulation beam 1354 to thus drive a respective articulation. Constant velocity joint 1380 spans longitudinally between distal retainer 1335 and proximal retainer 1332 to thus prevent rotation of end effector 1200 relative to shaft 1600 while also allowing articulation.

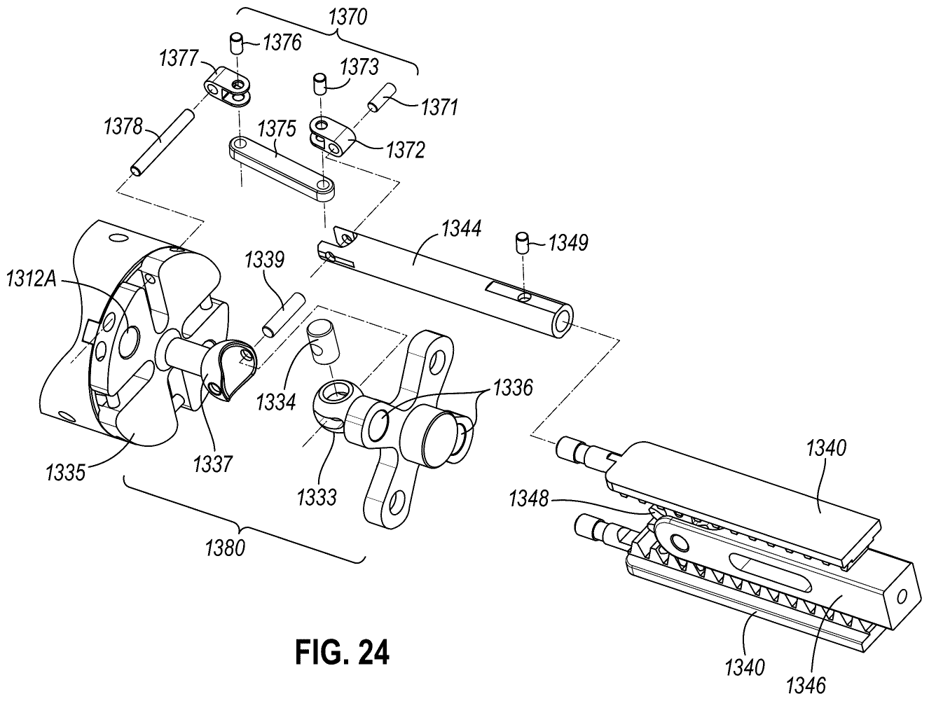

FIG. 24 shows an exploded view of a portion of articulation joint 1300 having a link assembly 1370 and constant velocity joint 1380. While only one link assembly 1370 is shown in FIG. 24, each of the other link assemblies 1370 not shown may be substantially identical in structure and function with respect to the corresponding yaw or pitch drive assembly. From proximal to distal, proximal link 1372 of link assembly 1370 is pivotably coupled to yaw articulation beam 1344 via a first pin 1371 such that proximal link 1372 can radially pivot relative to yaw articulation beam 1344. Proximal link 1372 is then pivotably coupled to middle link 1375 via second pin 1373 such that middle link 1375 can angularly pivot (e.g., in a circumferentially tangential direction) relative to proximal link 1372. Middle link 1375 is then pivotably coupled to distal link 1377 via a third pin 1376 such that distal link 1377 can angularly pivot relative to middle link 1375. Distal link 1377 is then pivotably coupled to distal retainer 1335 via a forth pin 1378 such that distal retainer 1335 can radially pivot relative to distal link 1377. Each of second pin 1373 and third pin 1376 may include an axis that intersects the longitudinal axis of shaft 1600 when surgical instrument 1100 is in the straight orientation. Each of first pin 1371 and fourth pin 1378 may be transverse to and offset from the longitudinal axis of shaft 1600 when surgical instrument 1100 is in the non-articulated, straight orientation.

Constant velocity joint 1380 includes a constant velocity ball 1333 coupled to proximal retainer 1332. Constant velocity ball 1333 includes an opening and a slot. A constant velocity shaft 1334 having a hole is sized to slide into the opening of constant velocity ball 1333. A constant velocity pin 1339 is then fitted inside the slot of constant velocity ball 1333 and the hole of constant velocity shaft 1334. Constant velocity shaft 1334 is thus rotatable within the opening of constant velocity ball 1333 and the rotation is bound by constant velocity pin 1339 being within the slot of constant velocity ball 1333. Constant velocity ball 1333 is retained within a constant velocity socket 1337 of distal retainer 1335 via constant velocity pin 1339. In this manner, distal retainer 1335 is rotationally fixed to proximal retainer 1332 while also being pivotable to thus allow articulation of end effector 1200 relative to shaft 1600 via articulation joint 1300.

Each yaw articulation beam 1344 may be rotationally coupled with the respective yaw rack gear 1340. A pin 1349 fixed to articulation beam 1344 may project into a central opening of articulation beam 1344. Pin 1349 may thereby engage a channel of yaw rack gear 1340 to allow for rotation of yaw articulation beam 1344 about its longitudinal axis relative to the yaw rack gear 1340 while also longitudinally fixing yaw articulation beam 1344 to yaw rack gear 1340. Similarly, each pitch articulation beam 1354 may be rotatable about its longitudinal axis relative to the respective pitch rack gear 1350. Such rotatability of articulation beams 1344, 1354 relative to their respective rack gears, 1340, 1350 enables angular displacement of links 1372, 1375, 1377 as articulation beams 1344, 1354 actuate proximally and distally, thereby protecting against binding of articulation joint 1300 during articulation about the yaw axis and/or the pitch axis.

Proximal retainer 1332 includes two laterally opposed firing openings 1336 and distal retainer 1335 includes two laterally opposed firing openings 1312. Each of firing openings 1336 are longitudinally aligned with firing openings 1312 while surgical instrument 1100 is in a straight orientation. Firing openings 1312, 1336 are sized to allow for translation of first push rod 504 and second push rod 506 as described above during staple firing. First push rod 504 and second push rod 506 are capable of translating through firing openings 1312, 1336 while surgical instrument 1100 is articulated to thus fire the staples. Proximal retainer 1332 includes two additional holes 90 degrees from firing openings 1336 which are used to fasten proximal retainer 1332 to shaft 1600.

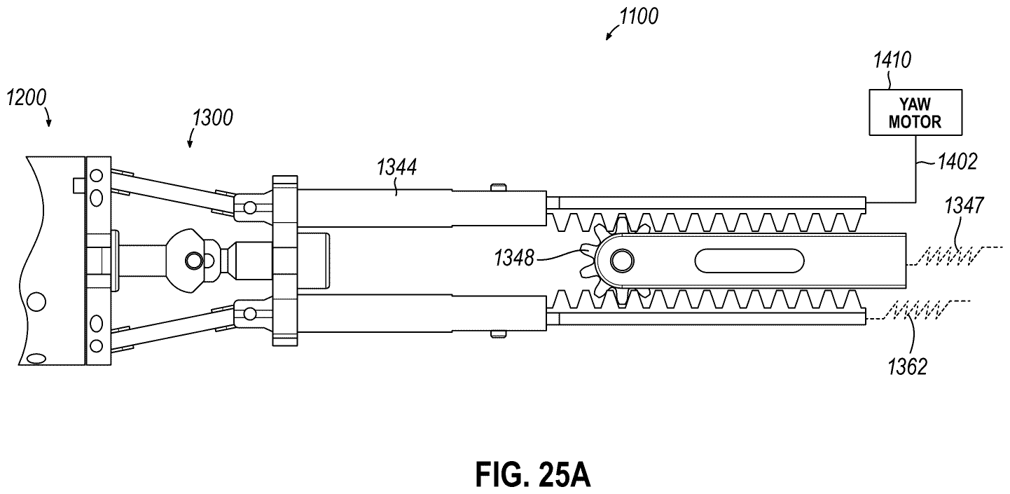

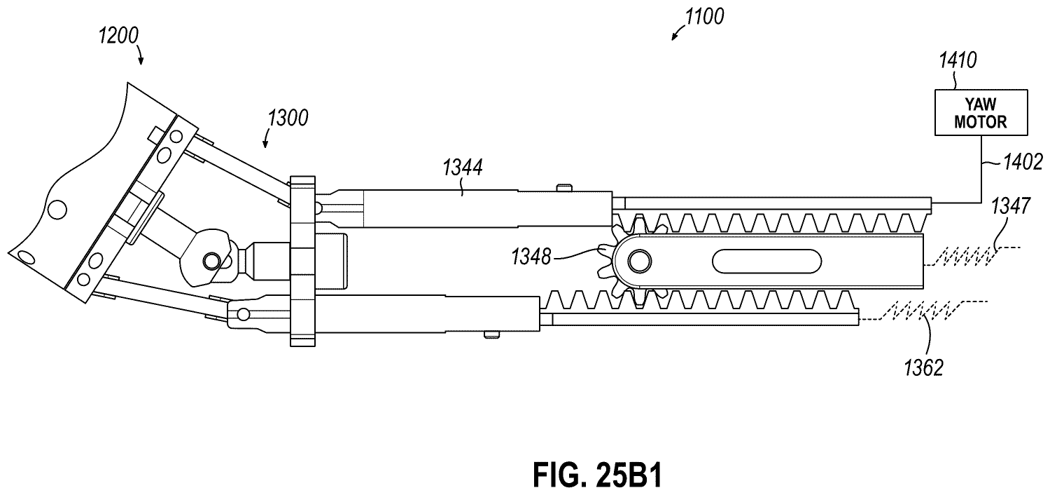

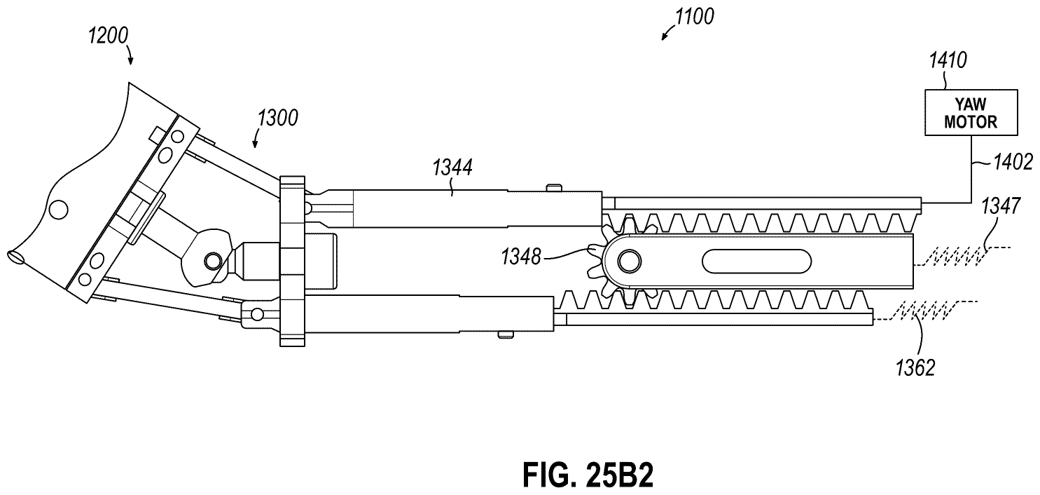

FIGS. 25A-25B2 show illustrative articulation of end effector 1200. FIG. 25A shows end effector 1200 and articulation joint 1300 in a straight orientation where both yaw rack gears 1340 are longitudinally aligned with each other to thus result in no articulation. Drive coupling 1402 is shown pulling on the upper depicted yaw rack gear 1340 to thus equal a biasing force being applied to the lower depicted yaw rack gear 1340 by spring 1362. As discussed below, spring 1347 may be operably positioned between yaw carrier 1346 and shaft 1600 to thus bias yaw carrier 1346 longitudinally.

FIGS. 25B1 and 25B2 depict different configurations of surgical instrument 1100 that provide in slightly different articulated orientations of end effector 1200, as described below. As mentioned above, yaw carrier 1346 and/or pitch carrier 1356 may be longitudinally fixed (see FIG. 25B1) or longitudinally translatable (i.e., floating) (see FIG. 25B2) relative to shaft 1600. For instance, yaw carrier 1346 and/or pitch carrier 1356 may be selectively fixed or permitted to float in response to an input provided by an operator. FIG. 25B 1 shows a length-conservative configuration of surgical instrument 1100 in which yaw carrier 1346 is fixed relative to shaft 1600. In this configuration, for any distance that upper depicted yaw rack gear 1340 translates longitudinally, lower depicted yaw rack gear 1340 translates longitudinally by the same distance in an opposing direction. When yaw carrier 1346 is fixed relative to shaft 1600, spring 1347 may be omitted.

FIG. 25B2 shows a non-length-conservative configuration of surgical instrument 1100 in which yaw carrier 1346 is longitudinally translatable (i.e. floating) relative to shaft 1600. In other words, yaw carrier 1346 is capable of translating parallel to a longitudinal axis of shaft 1600. Upper depicted yaw rack gear 1340, yaw carrier 1346, and lower depicted yaw rack gear 1340 may cach move different longitudinal amounts from one another such that upper depicted yaw rack gear 1340 is shown having moved a greater proximal distance than a distal distance moved by lower depicted yaw rack gear 1340. Yaw carrier 1346 and yaw pinion gear 1348 are shown having cach moved proximally a distance that is less the proximal distance of upper depicted yaw rack gear 1340. Spring 1347 may be used to bias yaw carrier 1346 distally. As described throughout, the pitch drive assembly may be substantially similar to the yaw drive assembly in structure and function but has not been shown for brevity and clarity.

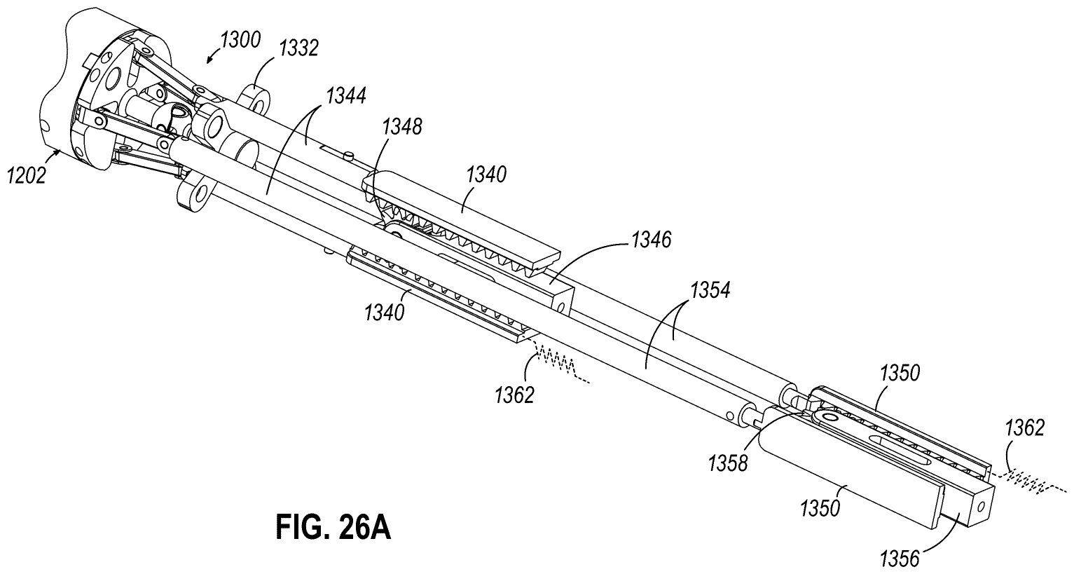

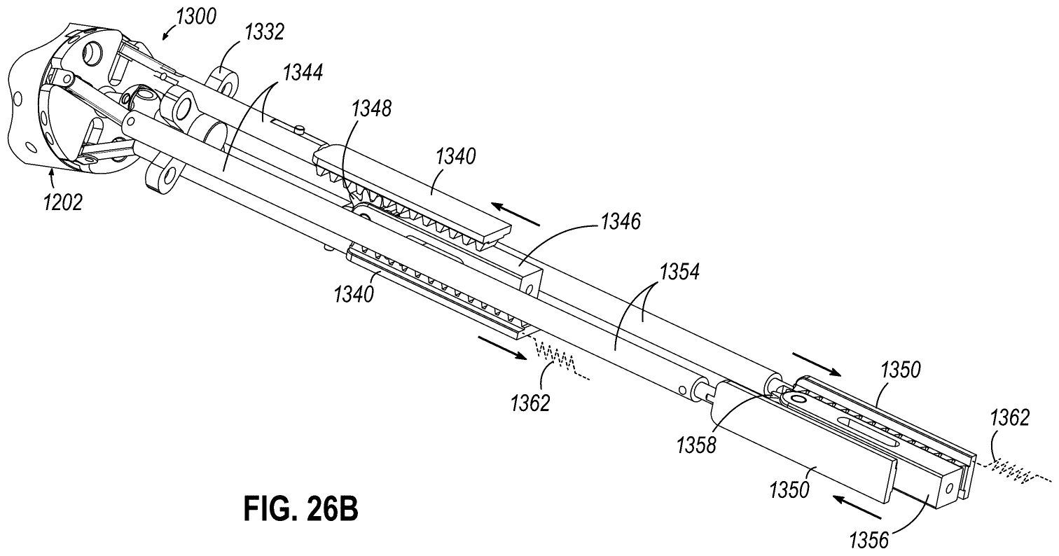

FIGS. 26A-26B shows articulation of surgical instrument 1100 in both pitch and yaw directions. FIG. 26A shows end effector 1200 and articulation joint 1300 in the straight, non-articulated orientation where each pairs of yaw rack gears 1340 and pitch rack gears 1350 are longitudinally aligned. FIG. 26B shows the driven yaw rack gear 1340 being pushed distally by yaw motor 1410 (see FIG. 21) while the non-driven yaw rack gear 1340 advances proximally to thus drive end effector 1200 about the yaw axis into the depicted downward orientation. Simultaneously, the driven pitch rack gear 1350 is pushed distally by pitch motor 1412 (see FIG. 21) while the non-driven pitch rack gear 1350 advances proximally to thus drive end effector 1200 about the pitch axis towards the right (into the page). During this articulation, constant velocity joint 1380 inhibits rolling of end effector 1200 relative to shaft 1600 about the longitudinal axis of shaft 1600. The combined articulations about the yaw and pitch axes result in end effector 1200 being oriented down and to the right relative to the longitudinal axis of shaft 1600.

While FIG. 26B shows articulation of surgical instrument 1100 in both pitch and yaw directions, it is possible for articulation to occur in only a pitch or a yaw direction. In such a situation, each of link assemblies 1370 will articulate. As a non-limiting example, articulation in a pitch direction will articulate pitch link assemblies 1370 as described above and will also articulate yaw link assemblies 1370 as they inhibit articulation of end effector 1200 in the yaw direction.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument (1100), comprising:
(a) a shaft (1600) defining a longitudinal axis;
(b) an end effector (1200); and
(c) an articulation assembly (1300) positioned between the shaft and the end effector and configured to articulate the end effector relative to the shaft about each of a pitch axis and a yaw axis, the articulation assembly comprising:
   (i) a pitch articulation beam (1354), wherein the pitch articulation beam is configured to drive a pitch of the end effector relative to the shaft about the pitch axis by pushing a first linked portion of the end effector distally and pulling the first linked portion of the end effector proximally, and
   (ii) a yaw articulation beam (1344), wherein the yaw articulation beam is configured to drive a yaw of the end effector relative to the shaft about the yaw axis by pushing a second linked portion of the end effector distally and pulling the second linked portion of the end effector proximally.

### Example 2

The surgical instrument of example 1, the articulation assembly further comprising:
(i) a pivot joint (1380) configured to inhibit rolling of the end effector relative to the shaft about the longitudinal axis while permitting articulation of the end effector relative to the longitudinal axis about each of the pitch axis and the yaw axis;
(ii) a first link assembly (1370) including four hinge joints (1371, 1373, 1376, 1378), the first link assembly being configured to drive the pitch of the end effector relative to the shaft; and
(iii) a second link assembly (1370) including four hinge joints (1371, 1373, 1376, 1378), the second link assembly being configured to drive the yaw of the end effector relative to the shaft,
wherein the four hinge joints of each of the first and second link assemblies are collectively configured to pivot during a combined pitch and yaw motion of the end effector relative to the shaft,
wherein the first link assembly is coupled to the pitch articulation beam, and
wherein the second link assembly is coupled to the yaw articulation beam.

### Example 3

The surgical instrument of example 2, wherein the surgical instrument is a surgical stapler, wherein the end effector includes an anvil jaw (1204) and a cartridge jaw (1202), wherein the anvil jaw is pivotable relative to the cartridge jaw, wherein each of the first link assembly and the second link assembly is pivotably coupled to the cartridge jaw to thereby drive the respective pitch and yaw of the end effector.

### Example 4

The surgical instrument of example 2, wherein the pitch articulation beam and a portion of the first link assembly are rotatable relative to the shaft.

### Example 5

The surgical instrument as in one of examples 2-4, wherein the second link assembly is configured to articulate while inhibiting a yaw articulation during a sole pitch articulation of the end effector by the first link assembly.

### Example 6

The surgical instrument as in one of examples 2-5, wherein each of two hinge joints of the four hinge joints of each of the first and second link assemblies includes a hinge axis that intersects the longitudinal axis.

### Example 7

The surgical instrument as in one of examples 2-6, the articulation assembly further comprising a first motor (1412) coupled to the first link assembly and operable to thereby drive the pitch of the end effector, the articulation assembly further comprising a second motor (1410) coupled to the second link assembly and operable to thereby drive the yaw of the end effector.

### Example 8

The surgical instrument as in one of examples 2-7, the articulation assembly further comprising:
(a) a third link assembly (1370) including four hinge joints (1371, 1373, 1376, 1378), the third link assembly being configured to drive the pitch of the end effector relative to the shaft; and
(b) a fourth link assembly (1370) including four hinge joints (1371, 1373, 1376, 1378), the fourth link assembly being configured to drive the yaw of the end effector relative to the shaft.

### Example 9

The surgical instrument of example 8, further comprising:
(a) a first rack gear (1350) coupled to the first link assembly;
(b) a second rack gear (1350) coupled to the third link assembly; and
(c) a first pinion gear (1358) meshed between the first rack gear and the second rack gear and thereby configured to drive the third link assembly in an opposing direction to the first link assembly.

### Example 10

The surgical instrument of example 9, wherein the first pinion gear is longitudinally fixed relative to the shaft.

### Example 11

The surgical instrument of example 9, wherein the first pinion gear is longitudinally translatable relative to the shaft.

### Example 12

The surgical instrument as in one of examples 2-11, wherein the pivot joint comprises a constant velocity joint.

### Example 13

The surgical instrument as in one of examples 2-12, wherein the pivot joint includes a ball (1333), a socket (1337), and a pin (1378), wherein the ball is positioned at least partially within the socket, wherein the pin is positioned within a slot of the ball and is affixed to the socket to thereby inhibit rotation of the ball relative to the socket.

### Example 14

The surgical instrument as in any of examples 1-2 or 4-13, wherein the end effector includes a pair of jaws configured to cooperate to clamp and staple tissue with a plurality of staples, wherein the surgical instrument includes a knife configured to advance distally through the end effector to approximate the jaws and thereby clamp the tissue and also cut the tissue.

### Example 15

The surgical instrument as in one of examples 1-14, wherein the pitch articulation beam comprises a first pitch articulation beam and the yaw articulation beam comprises a first yaw articulation beam, the articulation assembly further comprising a second pitch articulation beam extending parallel to the first pitch articulation beam and a second yaw articulation beam extending parallel to the second pitch articulation beam.

The following clauses also relate to various non-exhaustive ways in which the teachings herein may be combined or applied.
1. A surgical instrument, comprising:
   (a) a shaft defining a longitudinal axis;
   (b) an end effector; and
   (c) an articulation assembly positioned between the shaft and the end effector and configured to articulate the end effector relative to the shaft about each of a pitch axis and a yaw axis, the articulation assembly comprising:
      (i) a pitch articulation beam, wherein the pitch articulation beam is configured to drive a pitch of the end effector relative to the shaft about the pitch axis by pushing a first linked portion of the end effector distally and pulling the first linked portion of the end effector proximally, and
      (ii) a yaw articulation beam, wherein the yaw articulation beam is configured to drive a yaw of the end effector relative to the shaft about the yaw axis by pushing a second linked portion of the end effector distally and pulling the second linked portion of the end effector proximally.
2. The surgical instrument of clause 1, the articulation assembly further comprising:
   (i) a pivot joint configured to inhibit rolling of the end effector relative to the shaft about the longitudinal axis while permitting articulation of the end effector relative to the longitudinal axis about each of the pitch axis and the yaw axis;
   (ii) a first link assembly including four hinge joints, the first link assembly being configured to drive the pitch of the end effector relative to the shaft; and
   (iii) a second link assembly including four hinge joints, the second link assembly being configured to drive the yaw of the end effector relative to the shaft,
   wherein the four hinge joints of each of the first and second link assemblies are collectively configured to pivot during a combined pitch and yaw motion of the end effector relative to the shaft,
   wherein the first link assembly is coupled to the pitch articulation beam, and
   wherein the second link assembly is coupled to the yaw articulation beam.
3. The surgical instrument of clause 2, wherein the surgical instrument is a surgical stapler, wherein the end effector includes an anvil jaw and a cartridge jaw, wherein the anvil jaw is pivotable relative to the cartridge jaw, wherein each of the first link assembly and the second link assembly is pivotably coupled to the cartridge jaw to thereby drive the respective pitch and yaw of the end effector.
4. The surgical instrument of clause 2, wherein the pitch articulation beam and a portion of the first link assembly are rotatable relative to the shaft.
5. The surgical instrument of clause 2, wherein the second link assembly is configured to articulate while inhibiting a yaw articulation during a sole pitch articulation of the end effector by the first link assembly.
6. The surgical instrument of clause 2, wherein each of two hinge joints of the four hinge joints of each of the first and second link assemblies includes a hinge axis that intersects the longitudinal axis.
7. The surgical instrument of clause 2, the articulation assembly further comprising a first motor coupled to the first link assembly and operable to thereby drive the pitch of the end effector, the articulation assembly further comprising a second motor coupled to the second link assembly and operable to thereby drive the yaw of the end effector.
8. The surgical instrument of clause 2, the articulation assembly further comprising:
   (a) a third link assembly including four hinge joints, the third link assembly being configured to drive the pitch of the end effector relative to the shaft; and
   (b) a fourth link assembly including four hinge joints, the fourth link assembly being configured to drive the yaw of the end effector relative to the shaft.
9. The surgical instrument of clause 8, further comprising:
   (a) a first rack gear coupled to the first link assembly;
   (b) a second rack gear coupled to the third link assembly; and
   (c) a first pinion gear meshed between the first rack gear and the second rack gear and thereby configured to drive the third link assembly in an opposing direction to the first link assembly.
10. The surgical instrument of clause 9, wherein the first pinion gear is longitudinally fixed relative to the shaft.
11. The surgical instrument of clause 9, wherein the first pinion gear is longitudinally translatable relative to the shaft.
12. The surgical instrument of clause 2, wherein the pivot joint comprises a constant velocity joint.
13. The surgical instrument of clause 2, wherein the pivot joint includes a ball, a socket, and a pin, wherein the ball is positioned at least partially within the socket, wherein the pin is positioned within a slot of the ball and is affixed to the socket to thereby inhibit rotation of the ball relative to the socket.
14. The surgical instrument of clause 2, wherein the end effector includes a pair of jaws configured to cooperate to clamp and staple tissue with a plurality of staples, wherein the surgical instrument includes a knife configured to advance distally through the end effector to approximate the jaws and thereby clamp the tissue and also cut the tissue.
15. The surgical instrument of clause 14, further comprising a pair of pushers that longitudinally span the articulation assembly, wherein the pushers are configured to drive the knife proximally and distally through the end effector.
16. A surgical stapler, comprising:
   (a) a shaft defining a longitudinal axis;
   (b) an end effector; and
   (c) an articulation assembly positioned between the shaft and the end effector and configured to articulate the end effector relative to the shaft, the articulation assembly comprising:
      (i) a pivot joint positioned along the longitudinal axis and operatively coupling the shaft to the end effector; and
      (ii) a link assembly including a distal link, a proximal link, and a middle link, wherein the distal link is pivotable relative to each of the end effector and the middle link, wherein the proximal link is pivotable relative to each of the shaft and the middle link.
17. A surgical instrument, comprising:
   (a) a shaft defining a longitudinal axis;
   (b) an end effector; and
   (c) an articulation assembly positioned between the shaft and the end effector and configured to articulate the end effector relative to the shaft, the articulation assembly comprising:
      (i) a pitch drive assembly configured to drive a pitch angle of the end effector relative to the shaft, the pitch drive assembly including:
         (A) a pair of pitch articulation beams, wherein each pitch articulation beam is configured to translate parallel to the longitudinal axis, and
         (B) a pair of pitch link assemblies, wherein each pitch link assembly is coupled to a respective pitch articulation beam and to the end effector; and
      (ii) a yaw drive assembly configured to drive a yaw angle of the end effector relative to the shaft, the pitch drive assembly including:
         (A) a pair of yaw articulation beams, wherein each yaw articulation beam is configured to translate parallel to the longitudinal axis, and
         (B) a pair of yaw link assemblies, wherein each yaw link assembly is coupled to a respective yaw articulation beam and to the end effector.
18. The surgical instrument of clause 17, wherein the pitch articulation beams and the yaw articulation beams are spaced apart circumferentially at 90 degree increments about the longitudinal axis.
19. The surgical instrument of clause 18, wherein the pitch articulation beams are diametrically opposed from one another about the longitudinal axis and are rotatable within the shaft.
20. The surgical instrument of clause 17, where each of the pitch link assemblies and each of the yaw link assemblies includes two radially pivotable hinge joints and two circumferentially pivotable hinge joints.

### IV. Miscellaneous

It should be understood that any one or more of the teachings, expressions, versions, examples, etc. described herein may be combined with any one or more of the other teachings, expressions, versions, examples, etc. that are described herein. The above-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as those made available by Auris Health, Inc. of Redwood City, CA or by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1.-15. (canceled)

16. A surgical instrument, comprising:
(a) a shaft defining a longitudinal axis;
(b) an end effector; and
(c) an articulation assembly positioned between the shaft and the end effector and configured to articulate the end effector relative to the shaft about each of a pitch axis and a yaw axis, the articulation assembly comprising:
(i) a pitch articulation beam and a carrier operably engaged with the pitch articulation beam, wherein the pitch articulation beam is configured to drive a pitch of the end effector relative to the shaft about the pitch axis by pushing a first linked portion of the end effector distally and pulling the first linked portion of the end effector proximally, wherein the carrier is configured to maintain a fixed configuration relative to the shaft such that the carrier is fixed relative to the shaft during an articulation of the end effector, and
(ii) a yaw articulation beam, wherein the yaw articulation beam is configured to drive a yaw of the end effector relative to the shaft about the yaw axis by pushing a second linked portion of the end effector distally and pulling the second linked portion of the end effector proximally.

17. The surgical instrument of claim 16, the articulation assembly further comprising:
(i) a pivot joint configured to inhibit rolling of the end effector relative to the shaft about the longitudinal axis while permitting articulation of the end effector relative to the longitudinal axis about each of the pitch axis and the yaw axis;
(ii) a first link assembly including four hinge joints, the first link assembly being configured to drive the pitch of the end effector relative to the shaft; and
(iii) a second link assembly including four hinge joints, the second link assembly being configured to drive the yaw of the end effector relative to the shaft,
wherein the four hinge joints of each of the first and second link assemblies are collectively configured to pivot during a combined pitch and yaw motion of the end effector relative to the shaft,
wherein the first link assembly is coupled to the pitch articulation beam, and
wherein the second link assembly is coupled to the yaw articulation beam.

18. The surgical instrument of claim 17, wherein the surgical instrument is a surgical stapler, wherein the end effector includes an anvil jaw and a cartridge jaw, wherein the anvil jaw is pivotable relative to the cartridge jaw, wherein each of the first link assembly and the second link assembly is pivotably coupled to the cartridge jaw to thereby drive the respective pitch and yaw of the end effector.

19. The surgical instrument of claim 17, wherein the pitch articulation beam and a portion of the first link assembly are rotatable relative to the shaft.

20. The surgical instrument of claim 17, wherein the second link assembly is configured to articulate while inhibiting a yaw articulation during a sole pitch articulation of the end effector by the first link assembly.

21. The surgical instrument of claim 17, wherein each of two hinge joints of the four hinge joints of each of the first and second link assemblies includes a hinge axis that intersects the longitudinal axis.

22. The surgical instrument of claim 17, the articulation assembly further comprising a first motor coupled to the first link assembly and operable to thereby drive the pitch of the end effector, the articulation assembly further comprising a second motor coupled to the second link assembly and operable to thereby drive the yaw of the end effector.

23. The surgical instrument of claim 17, the articulation assembly further comprising:
(a) a third link assembly including four hinge joints, the third link assembly being configured to drive the pitch of the end effector relative to the shaft; and
(b) a fourth link assembly including four hinge joints, the fourth link assembly being configured to drive the yaw of the end effector relative to the shaft.

24. The surgical instrument of claim 23, further comprising:
(a) a first rack gear coupled to the first link assembly;
(b) a second rack gear coupled to the third link assembly; and
(c) a first pinion gear meshed between the first rack gear and the second rack gear and thereby configured to drive the third link assembly in an opposing direction to the first link assembly.

25. The surgical instrument of claim 16, the articulation assembly further comprising a spring operably coupled between the carrier and the shaft to thereby bias the carrier relative to the shaft.

26. The surgical instrument of claim 24, wherein the first pinion gear is longitudinally translatable relative to the shaft.

27. The surgical instrument of claim 17, wherein the pivot joint comprises a constant velocity joint.

28. The surgical instrument of claim 17, wherein the pivot joint includes a ball, a socket, and a pin, wherein the ball is positioned at least partially within the socket, wherein the pin is positioned within a slot of the ball and is affixed to the socket to thereby inhibit rotation of the ball relative to the socket.

29. The surgical instrument of claim 17, wherein the end effector includes a pair of jaws configured to cooperate to clamp and staple tissue with a plurality of staples, wherein the surgical instrument includes a knife configured to advance distally through the end effector to approximate the jaws and thereby clamp the tissue and also cut the tissue.

30. The surgical instrument of claim 16, wherein the carrier is configured to selectively transition between a floating configuration and the fixed configuration relative to the shaft.

31. A surgical stapler, comprising:
(a) a shaft defining a longitudinal axis;
(b) an end effector; and
(c) an articulation assembly positioned between the shaft and the end effector and configured to articulate the end effector relative to the shaft, the articulation assembly comprising:
(i) a link assembly including a distal link, a proximal link, and a middle link, wherein the distal link is pivotable relative to each of the end effector and the middle link, wherein the proximal link is pivotable relative to each of the shaft and the middle link; and
(ii) a drive assembly including a first portion and a second portion operably coupled to the link assembly, wherein the drive assembly is configured to drive an articulation of the end effector, wherein the drive assembly is configured to selectively transition between a length-conservative configuration and a non-length-conservative configuration, wherein, in the length-conservative configuration, each of the first portion and the second portion move an equal distance upon an articulation, wherein, in the non-length-conservative configuration, each of the first portion and the second portion move an unequal distance upon an articulation.

32. A surgical instrument, comprising:
(a) a shaft defining a longitudinal axis;
(b) an end effector; and
(c) an articulation assembly positioned between the shaft and the end effector and configured to articulate the end effector relative to the shaft, the articulation assembly comprising:
(i) a pitch drive assembly configured to drive a pitch angle of the end effector relative to the shaft, the pitch drive assembly including:
(A) a pair of pitch articulation beams, wherein each pitch articulation beam is configured to translate parallel to the longitudinal axis, where each pitch articulation beam of the pair of pitch articulation beams is configured to translate independently of the other pitch articulation beam, and
(B) a pair of pitch link assemblies, wherein each pitch link assembly is coupled to a respective pitch articulation beam and to the end effector; and
(ii) a yaw drive assembly configured to drive a yaw angle of the end effector relative to the shaft, the yaw drive assembly including:
(A) a pair of yaw articulation beams, wherein each yaw articulation beam is configured to translate parallel to the longitudinal axis, and
(B) a pair of yaw link assemblies, wherein each yaw link assembly is coupled to a respective yaw articulation beam and to the end effector.

33. The surgical instrument of claim 32, wherein the pitch articulation beams and the yaw articulation beams are spaced apart circumferentially at 90 degree increments about the longitudinal axis.

34. The surgical instrument of claim 33, wherein the pitch articulation beams are diametrically opposed from one another about the longitudinal axis and are rotatable within the shaft.

35. The surgical instrument of claim 32, where each of the pitch link assemblies and each of the yaw link assemblies includes two radially pivotable hinge joints and two circumferentially pivotable hinge joints.
